(19) United States
(12) Patent Application Publication
(10) Pub. No.: US 2014/0339088 A1
(43) Pub. Date: Nov. 20, 2014

(54) DIELECTROPHORESIS METHODS FOR DETERMINING A PROPERTY OF A PLURALITY OF CANCER CELLS

(71) Applicants: Eva M. Schmelz, Blacksburg, VA (US); Elizabeth S. Elvington, Blacksburg, VA (US); Alireza Salmanzadeh, Blacksburg, VA (US); Mike Sano, Durham, NC (US); Paul C. Roberts, Blacksburg, VA (US); Rafael V. Davalos, Blacksburg, VA (US)

(72) Inventors: Eva M. Schmelz, Blacksburg, VA (US); Elizabeth S. Elvington, Blacksburg, VA (US); Alireza Salmanzadeh, Blacksburg, VA (US); Mike Sano, Durham, NC (US); Paul C. Roberts, Blacksburg, VA (US); Rafael V. Davalos, Blacksburg, VA (US)

(73) Assignee: VIRGINIA TECH INTELLECTUAL PROPERTIES, INC., Blacksburg, VA (US)

(21) Appl. No.: 14/449,050

(22) Filed: Jul. 31, 2014

Related U.S. Application Data
(63) Continuation-in-part of application No. 13/269,286, filed on Oct. 7, 2011.
Application No. 13/269,286 is a continuation-in-part of application No. 12/720,406, filed on Mar. 9, 2010.
(60) Provisional application No. 61/860,309, filed on Jul. 31, 2013.
(60) Provisional application No. 61/390,748, filed on Oct. 7, 2010.
(60) Provisional application No. 61/158,553, filed on Mar. 9, 2009.
(60) Provisional application No. 61/252,942, filed on Oct. 19, 2009.

(51) Int. Cl. G01N 33/487; G01N 27/447; G01N 33/50

(52) U.S. Cl. CPC G01N 33/48735; G01N 33/5023; G01N 33/5011; G01N 27/44704
USPC 204/547

(57) ABSTRACT

Provided are dielectrophoresis (DEP) devices and methods that allow cell sorting to identify, isolate, and/or separate cells of interest based on electrical and physical properties of the cells. Particularly, provided are systems and methods for manipulating particles suspended in a fluid, e.g., cells, micro- or nano-particles, using their electrical signatures. Such methods can be performed using DEP, iDEP, and/or cDEP (contactless dielectrophoresis, where direct contact between the electrodes and the sample is avoided). Typically, an electric field is induced in a sample comprising the target particles and/or cells, such as cancer cells, and the spatial distribution of cells is measured to identify one or more characteristics or properties of the cancer cells. The identified characteristics of the sorted cells can be used to determine drug efficacy and/or resistance with respect to the cells.

### CROSS-REFERENCE TO RELATED APPLICATIONS

This application relies on the disclosure of and claims priority to and the benefit of the filing date of U.S. Provisional Patent Application No. 61/860,309, filed on Jul. 31, 2013, and this application is a Continuation-In-Part application of U.S. application Ser. No. 13/269,286, filed on Oct. 7, 2011, which claims priority to and the benefit of the filing date of U.S. Provisional Patent Application No. 61/390,748, filed on Oct. 7, 2010 and which is also a Continuation-In-Part Application of U.S. application Ser. No. 12/720,406 filed on Mar. 9, 2010, which claims priority to and the benefit of the filing date of U.S. Provisional Patent Application No. 61/158,553, filed on Mar. 9, 2009 and U.S. Provisional Patent Application No. 61/252,942, filed on Oct. 19, 2009, the disclosures of which are hereby incorporated by reference herein each in its entirety.

### BACKGROUND OF THE INVENTION

1. Field of the Invention

The present disclosure relates to devices and methods for dielectrophoresis (DEP) for manipulation of cells or particles. More particularly, the present disclosure relates to methods for determining one or more intrinsic electrical properties of a plurality of cancer cells through dielectrophoresis, such as by using contactless dielectrophoresis.

2. Description of Related Art

Ovarian cancer is the most common cause of death from gynecological malignancies and one of the top causes of cancer-related deaths of women in the United States and Europe [1, 2]. The relative 5-year survival rate for invasive epithelial ovarian cancer patients diagnosed at early stages is more than 90%, but drops to less than 30% when the disease is first diagnosed at later stages [3A], highlighting the importance of early detection and the need for improved treatment options.

Chemotherapeutic regimens typically conform to institutional protocols and subjective experiences of individual physicians, however, responses to these protocols can vary greatly between patients. This is largely due to the difficulty in assessing an individual's response to a particular regimen prior to treatment. More than 65% of women diagnosed with Stage III or later ovarian cancer will experience recurrence of the disease and will need to undergo additional rounds of chemotherapy [4A]. Chemotherapeutic resistance, a common occurrence in progressive disease [5A], can limit the success of consecutive rounds of therapy in the treatment of any type of cancer. There are a number of pharmacological, physiological, and molecular mechanisms associated with this resistance, however, the exact mechanisms which cause resistance are still unresolved. Interestingly, recent investigations have shown that drug resistance can occur at an early point in the tumorigenic pathway, prior to full malignant transformation [5A]. Thus, there is a need in the art for more effective identification of drug resistance and sensitivity and other properties of cancer cells, as well as early detection methods.

Ovarian cancer is a heterogeneous disease with a broad genetic and epigenetic profile, various histological manifestations and even different origins. Current conventional chemotherapeutic drugs targeting oncogenes such as Her-2 or VEGFR are successful in less than 30% of patients with aberrant expression of these targets, require chemotherapeutic dosages that are highly toxic to non-transformed cells, and/or induce drug resistance in a sub-population of cancer cells causing disease recurrence within 5-6 months. This heterogeneity would require the determination of the individual geno/phenotype for more successful treatment decisions that can enhance the survival of afflicted women.

The inventors have shown in this disclosure that the intrinsic electrical properties or “bioelectric signature” (for example, as measured by the dielectrophoretic response) of murine ovarian epithelial cells changes during cancer progression [6A], allowing for a clear distinction of early (pre-malignant) and late stage cancer cells from resident peritoneal cells [7A]. Preliminary findings indicate that these properties are also altered in tumor-initiating (TIC) or stem cell-like populations and by sphingolipid metabolites that either enhance or inhibit cancer growth [8A]. Whether the bioelectrical signature of TICs can be exploited to identify this population in primary tumors and/or metastases is currently unknown. Thus, the bioelectrical properties of cancer cells will allow for detection of early stages of ovarian cancer independent of specific molecular markers. Hence, the bioelectrical signature may be unique to the cancer cell and/or its stem-like population, and the response to treatment. It is thus expected that the drug resistant phenotype of cells will impart a unique bioelectrical signature (otherwise referred to as a bioelectrical fingerprint in this disclosure) that will enable early identification of drug resistant cells that may also be exploited to monitor the development of drug resistance during tumor recurrence.

The bioelectrical signature of cells can be determined by dielectrophoresis (DEP), the motion of a polarizable particle in a non-uniform electric field [9A, 10A]. DEP is successful technique for nondestructive manipulation [11], separation [12A], and detection [13A] of bioparticles in microdevices, based on their size, shape, internal structure, and electrical properties such as conductivity and polarizability. In contrast to techniques such as fluorescence activated and magnetic cell sorting [14A, 15A], DEP does not require the use of target-specific antibodies and has been shown to successfully isolate cancer cells from blood [16A, 17A, 18A], and from CD34+ hematopoietic stem cells [19A]. Thus, DEP could overcome the limitation of current approaches and provide a platform to assess individual drug responsiveness and metastatic potential. The inventors envision that this approach can be also applied to other cancer types and uses such as cancer cell detection.

### SUMMARY OF THE INVENTION

Embodiments of the present disclosure provide a method of determining a property of a plurality of cancer cells. In embodiments, the method comprises providing a dielectrophoresis device comprising a sample channel for receiving a sample. In preferred embodiments, the device comprises a sample channel or series of separating channels with a separating portion; a first electrode channel for receiving a first electrode; a first insulation barrier between the first electrode channel and the sample channel; a second electrode channel for receiving a second electrode; and a second insulation barrier between the second electrode channel and the sample channel. Other embodiments may not require a separate channel to contain the fluid portion.

The method comprises providing a plurality of cancer cells, such as in a cancer cell suspension, introducing the cancer cell suspension to the sample channel, generating an AC electric field through the first and second electrode, and measuring the spatial distribution of cells through the sample channel, wherein the spatial distribution is characteristic of a property of the cancer cells. The spatial distribution response can be used to identify and differentiate cancer cells from one another and/or characterize them, or interrogate, separate, or enrich them for further selective treatment of certain cells, such as downstream characterization protocols as needed, including for example, Genomics, Biochemical profiles, etc.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1 is graph showing crossover frequency for MOSE cells. The FFL stem cell electrical signature increases compared to the MOSE-L parent stage cells.

FIG. 2 is a schematic diagram showing a cDEP Device Schematic.

FIGS. 3A and 3B are images showing MOSE cells experiencing negative and positive DEP, respectively.

FIG. 4 is a diagram showing a summary of cancer cell separation studies.

FIG. 5 is a schematic diagram showing an overhead view schematic of an exemplary low frequency single-layer microfluidic device used for characterizing MOSE cells. The inset detail view shows a sawtooth feature and the thin insulating barrier separating sample channel and electrode channels.

FIG. 6A is a schematic diagram showing a surface plot for computational modeling of ∇(\vec{E}_{RMS}·\vec{E}_{RMS}) in the sample channel of a multilayer cDEP device with overlaid curved tapered electrode channels. The plot was generated at 100 V, 200 kHz. There are 4 electrode pairs, the included angle of the curves is 45′, and the nominal ratio of electrode channel width to gap width is 2:1.

FIG. 6B is a graph showing a plot along x-coordinates at z=35 μm, y=0 of the change in ∇(\vec{E}_{RMS}·\vec{E}_{RMS}) with x-position and frequency. The legend (units of Hz) shows the frequency range 100-500 kHz.

FIG. 7A is a schematic diagram showing a surface plot for computational modeling of ∇(\vec{E}_{RMS}·\vec{E}_{RMS}) in the sample channel of a multilayer cDEP device with curved tapered electrode channels.

FIG. 7B is a graph showing a line plot along the x-centerline of the sample channel demonstrating the change in ∇(\vec{E}_{RMS}·\vec{E}_{RMS}) with frequency. The legend shows the frequency value in Hz.

FIGS. 8A-C are illustrations showing computational modeling of samples channel: (a) shear rate, (b) electric field magnitude, and (c) ∇(\vec{E}_{RMS}·\vec{E}_{RMS}).

FIGS. 9A and 9B are schematic diagrams of predictions of the particles trajectories at (a) 5 kHz and (b) 20 kHz in red lines for 10 particles. Trajectories appear to diminish down the channel due to a simulation artifact that occurs when trajectories encounter a wall ∇(\vec{E}_{RMS}·\vec{E}_{RMS}) is also presented in the background. Darker areas indicate higher ∇(\vec{E}_{RMS}·\vec{E}_{RMS}). The scale bar represents 500 μm.

FIGS. 10A-C are images showing MOSE-L cell movement in the sample channel (a) in control conditions under no external electric field, due to applying 200 VRMS and (b) negative DEP force, 5 kHz and (c) positive DEP force, 30 kHz.

FIGS. 10D-F are plots showing normalized cells distribution corresponding to (d) control distribution in (a), (e) negative DEP from (b), and (f) positive DEP from (c).

FIGS. 11A and 11B are graphs showing that SL-treated late stage cells revert back to early stage based on their electrical signature. (a) f_{XO}/σₘ and (b) specific membrane capacitance of untreated, So-treated, and S1P-treated of MOSE-E and -L cells. *, and ** represent p<0.001, and 0.01, respectively (n=3 for treated cells and n=6 for untreated cells experiments).

FIGS. 12A-12C are graphs showing computational modeling of ∇(E·E) at z=35 μm (a) toward to of channel (y=200 μm), (b) at center of channel (y=0 μm), and (c) toward bottom of channel (y=−200 μm). ∇(E·E) increases with increasing y-position in the channel due to narrowing width of the electrode channels.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

The present disclosure provides methods, devices, and systems to manipulate micro-particles suspended in biological fluids (including solid tumors in suspension) using their electrical characteristics signatures. In preferred embodiments, such methods can be performed in the absence of direct contact between the electrodes and the sample. Contactless dielectrophoresis (cDEP) employs the simplified fabrication processes of iDEP yet lacks the problems associated with the electrode-sample contact as in DEP [40A]. Methods of the invention can also be used with DEP and/or iDEP. One difference between cDEP and DEP is that DEP uses a solid metal electrode that does not require a separate channel as cDEP does to contain the fluid portion. By way of background, the following are cited and incorporated by reference in their entities: Eker B, Meissner R, Bertsch A, Mehta K, Renaud P (2013) Label-Free Recognition of Drug Resistance via Impedimetric Screening of Breast Cancer Cells. PLoS ONE 8(3): e57423. doi:10.1371/journal.pone.0057423) and U.S. Pat. No. 7,678,256.

cDEP relies upon reservoirs filled with highly conductive fluid to act as electrodes and provide the necessary electric field. These reservoirs are placed adjacent to the main microfluidic channel and are separated from the sample by a thin barrier of a dielectric material. The application of a high-frequency electric field to the electrode reservoirs causes their capacitive coupling to the main channel and an electric field is induced across the sample fluid.

Similar to traditional DEP and iDEP, cDEP may exploit the varying geometry of the electrodes to create spatial non-uniformities in the electric field. By utilizing reservoirs filled with a highly conductive solution, rather than a separate thin film array, the electrode structures employed by cDEP can be fabricated in the same step as the rest of the device; hence the process is conducive to mass production [40A]. Various embodiments of the present disclosure provide devices and methods for performing cDEP, iDEP, and/or DEP, as well as methods for fabricating such devices.

In general, the present disclosure provides devices and methods that allow cell sorting to identify, isolate or otherwise enrich cells of interest based on electrical and physical properties of the target cells. Typically, an electric field is induced in a sample comprising the target cells. In embodiments, the cells are cancer cells, but any cell may be used. Alternatively, any particle of size which would fit down the channel such as DNA, RNA, cell particles, protein, viruses, and bio-markers can also be used. The electric field can be induced in a main sorting microchannel using electrodes inserted in a highly conductive solution which is isolated from the microchannel by thin insulating barriers. The insulating barriers exhibit a capacitive behavior and an electric field is produced in the isolated microchannel by applying an AC electric field. Although it is possible to design the device so that electrodes contact the sample fluid, a contactless approach is preferred in which the electrodes do not come into contact with the sample fluid inside the microchannel. With no contact between the electrodes and sample fluid, electrolysis, bubble formation, fouling and contamination can be reduced or eliminated. In addition, the electric field is focused in a confined region and has a much lower intensity than that found in traditional insulator-based dielectrophoresis, so heating within the sample channel is negligible and the likelihood of cell lysis is greatly reduced. The system can also be used for characterizing and sorting micro- or nanoparticles.

The present disclosure provides devices and methods that allow for the determination of a cancer cells sensitivity or resistance to a cancer therapeutic agent (e.g., chemotherapeutic drugs), or, alternatively, the determination of malignant properties of the cancer cells such as the stage of transformation, tumorigenicity and metastatic potential, and stem-like properties, based on the electrical and physical properties of the cancer cell.

Further, the present disclosure provides devices and methods that allow for the detection and enrichment of drug-resistant cells by their altered bioelectrical fingerprint independent of their specific genotype and origin. The devices and methods can be used to define and characterize “specific” drug responsiveness and drug resistance that can be used to prescreen tumor cells of a particular patient for resistance (for example, drug resistant populations display an altered bioelectrical profile) prior to beginning a treatment regimen on that patient, including conventional drugs and novel experimental drugs and treatments. The devices and methods can be used for monitoring of treatment for efficacy and direct further treatment decisions/modifications of treatment. Further, the bioelectrical fingerprint is phenotype dependent. Further, the present disclosure provides devices and methods that allow for characterization of cells by their bioelectrical fingerprint, including by their “dielectrophoretic response”. As used herein, “dielectrophoretic response” means the intrinsic electrical characteristics of a cell; dependent upon its size, shape morphology and topography, including the internal organelles (nucleus size, polidity, endoplasmic reticulum) and structures, thickness of the lipid bilayer or cell membrane, nucleus size endoplasmic reticulum, and ratio of internal structures to cytoplasm.

Still further, the devices and methods can be used to detect and enrich drug-treated cells (conventional drugs and novel) by their electrical fingerprint to evaluate treatment efficacy. The present inventors have shown that the anti-cancer sphingolipid metabolite sphingosine shifts the cells towards a more benign phenotype while the pro-tumorigenic metabolite sphingosine-1-phosphate shifts the bioelectrical fingerprint towards a more aggressive phenotype (Salmanzadeh et al., Integr Biol (Camb). 2013 June; 5(6): 843-852). The devices and methods can be used to evaluate the efficacy of conventional drugs and drug combinations and evaluate new compounds/drugs/treatments.

The devices and methods can be used to evaluate the sensitivity or resistance of a cancer cell to any chemotherapeutic drug or any anti-cancer agent. Non-limiting examples of agents that cancer cells can be evaluated for resistance or sensitivity to include Abiraterone Acetate, ABITREXATE (Methotrexate), ABRAXANE (Paclitaxel Albumin-stabilized Nanoparticle Formulation), ADCETRIS (Brentuximab Vedotin), Ado-Trastuzumab Emtansine, ADRIAMYCIN (Doxorubicin Hydrochloride), ADRUCIL (Fluorouracil), Afatinib Dimaleate, AFINITOR (Everolimus), ALDARA (Imiquimod), Aldesleukin, Alemtuzumab, ALIMTA (Pemetrexed Disodium), ALOXI (Palonosetron Hydrochloride), AMBOCHLORIN (Chlorambucil), AMBOCLORIN (Chlorambucil), Aminolevulinic Acid, Anastrozole, Aprepitant, AREDIA (Pamidronate Disodium), ARIMIDEX (Anastrozole), AROMASIN (Exemestane), ARRANON (Nelarabine), Arsenic Trioxide, ARZERRA (Ofatumumab), Asparaginase Erwinia chrysanthemi, AVASTIN (Bevacizumab), Axitinib, Azacitidine, Bendamustine Hydrochloride, Bevacizumab, Bexarotene, BEXXAR (Tositumomab and I 131 Iodine Tositumomab), Bleomycin, Bortezomib, BOSULIF (Bosutinib), Cabazitaxel, Cabozantinib-S-Malate, CAMPATH (Alemtuzumab), CAMPTOSAR (Irinotecan Hydrochloride), Capecitabine, Carboplatin, Carfilzomib, CEENU (Lomustine), CERUBIDINE (Daunorubicin Hydrochloride), Cetuximab, Chlorambucil, Cisplatin, CLAFEN (Cyclophosphamide), Clofarabine, COMETRIQ (Cabozantinib-S-Malate), COSMEGEN (Dactinomycin), Crizotinib, Cyclophosphamide, CYFOS (Ifosfamide), Cytarabine, Dabrafenib, Dacarbazine, DACOGEN (Decitabine), Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, EFUDEX (Fluorouracil), ELITEK (Rasburicase), ELLENCE (Epirubicin Hydrochloride), ELOXATIN (Oxaliplatin), Eltrombopag Olamine, EMEND (Aprepitant), Enzalutamide, Epirubicin Hydrochloride, ERBITUX (Cetuximab), Eribulin Mesylate, ERIVEDGE (Vismodegib), Erlotinib Hydrochloride, ERWINAZE (Asparaginase Erwinia chrysanthemi), Etoposide, Everolimus, EVISTA (Raloxifene Hydrochloride), Exemestane, FARESTON (Toremifene), FASLODEX (Fulvestrant), FEMARA (Letrozole), Filgrastim, FLUDARA (Fludarabine Phosphate), Fludarabine Phosphate, FLUOROPLEX (Fluorouracil), Fluorouracil, Folinic acid, FOLOTYN (Pralatrexate), Fulvestrant, Gefitinib, Gemcitabine Hydrochloride, Gemtuzumab Ozogamicin, GEMZAR (Gemcitabine Hydrochloride), GILOTRIF (Afatinib Dimaleate), GLEEVEC (Imatinib Mesylate), HALAVEN (Eribulin Mesylate), HERCEPTIN (Trastuzumab), HYCAMTIN (Topotecan Hydrochloride), Ibritumomab Tiuxetan, ICLUSIG (Ponatinib Hydrochloride), Ifosfamide, Imatinib Mesylate, Imiquimod, INLYTA (Axitinib), INTRON A (Recombinant Interferon Alfa-2b), Iodine 131 Tositumomab and Tositumomab, Ipilimumab, IRESSA (Gefitinib), Irinotecan Hydrochloride, ISTODAX (Romidepsin), Ixabepilone, JAKAFI (Ruxolitinib Phosphate), JEVTANA (Cabazitaxel), Kadcyla (Ado-Trastuzumab Emtansine), KEOXIFENE (Raloxifene Hydrochloride), KEPIVANCE (Palifermin), KYPROLIS (Carfilzomib), Lapatinib Ditosylate, Lenalidomide, Letrozole, Leucovorin Calcium, Leuprolide Acetate, Lomustine, LUPRON (Leuprolide Acetate, MARQIBO (Vincristine Sulfate Liposome), MATULANE (Procarbazine Hydrochloride), Mechlorethamine Hydrochloride, MEGACE (Megestrol Acetate), Megestrol Acetate, MEKINIST (Trametinib), Mercaptopurine, Mesna, METHAZOLASTONE (Temozolomide), Methotrexate, Mitomycin, MOZOBIL (Plerixafor), MUSTARGEN (Mechlorethamine Hydrochloride), MUTAMYCIN (Mitomycin C), MYLOSAR (Azacitidine), MYLOTARG (Gemtuzumab Ozogamicin), Nanoparticle Paclitaxel (Paclitaxel Albumin-stabilized Nanoparticle Formulation), NAVELBINE (Vinorelbine Tartrate), Nelarabine, NEOSAR (Cyclophosphamide), NEUPOGEN (Filgrastim), NEXAVAR (Sorafenib Tosylate), Nilotinib, NOLVADEX (Tamoxifen Citrate), NPLATE (Romiplostim), Ofatumumab, Omacetaxine Mepesuccinate, ONCASPAR (Pegaspargase), ONTAK (Denileukin Diftitox), Oxaliplatin, Paclitaxel, Paclitaxel Albumin-stabilized Nanoparticle Formulation, Palifermin, Palonosetron Hydrochloride, Pamidronate Disodium, Panitumumab, Pazopanib Hydrochloride, Pegaspargase, Peginterferon Alfa-2b, PEG-INTRON (Peginterferon Alfa-2b), Pemetrexed Disodium, Pertuzumab, PLATINOL (Cisplatin), PLATINOL-AQ (Cisplatin), Plerixafor, Pomalidomide, POMALYST (Pomalidomide), Ponatinib Hydrochloride, Pralatrexate, Prednisone, Procarbazine Hydrochloride, PROLEUKIN (Aldesleukin), PROLIA (Denosumab), PROMACTA (Eltrombopag Olamine), PROVENGE (Sipuleucel-T), PURINETHOL (Mercaptopurine), Radium 223 Dichloride, Raloxifene Hydrochloride, Rasburicas, Recombinant Interferon Alfa-2b, Regorafenib, REVLIMID (Lenalidomide), RHEUMATREX (Methotrexate), Rituximab, Romidepsin, Romiplostim, RUBIDOMYCIN (Daunorubicin Hydrochloride), Ruxolitinib Phosphat, Sipuleucel-T, Sorafenib Tosylate, SPRYCEL (Dasatinib), STIVARGA (Regorafenib), Sunitinib Malate, SUTENT (Sunitinib Malate), SYLATRON (Peginterferon Alfa-2b), SYNOVIR (Thalidomide), SYNRIBO (Omacetaxine Mepesuccinate), TAFINLAR (Dabrafenib), Tamoxifen Citrate, TARABINE PFS (Cytarabine), TARCEVA (Erlotinib Hydrochloride), TARGRETIN (Bexarotene), TASIGNA (Nilotinib), TAXOL (Paclitaxel), TAXOTERE (Docetaxel), TEMODAR (Temozolomide), Temozolomide, Temsirolimus, Thalidomide, TOPOSAR (Etoposide), Topotecan Hydrochloride, Toremifene, TORISEL (Temsirolimus), Tositumomab and I 131 Iodine Tositumomab, TOTECT (Dexrazoxane Hydrochloride), Trametinib, Trastuzumab, TREANDA (Bendamustine Hydrochloride), TRISENOX (Arsenic Trioxide), TYKERB (Lapatinib Ditosylate), Vandetanib, VECTIBIX (Panitumumab), VeIP, VELBAN (Vinblastine Sulfate), VELCADE (Bortezomib), VELSAR (Vinblastine Sulfate), Vemurafenib, VEPESID (Etoposide), VIADUR (Leuprolide Acetate), VIDAZA (Azacitidine), Vinblastine Sulfate, Vincristine Sulfate, Vinorelbine Tartrate, Vismodegib, VORAXAZE (Glucarpidase), Vorinostat, VOTRIENT (Pazopanib Hydrochloride), WELLCOVORIN (Leucovorin Calcium), XALKORI (Crizotinib), XELODA (Capecitabine), XGEVA (Denosumab), XOFIGO (Radium 223 Dichloride), XTANDI (Enzalutamide), YERVOY (Ipilimumab), ZALTRAP (Ziv-Aflibercept), ZELBORAF (Vemurafenib), ZEVALIN (Ibritumomab Tiuxetan), ZINECARD (Dexrazoxane Hydrochloride), Ziv-Aflibercept, Zoledronic Acid, ZOLINZA (Vorinostat), ZOMETA (Zoledronic Acid), and ZYTIGA (Abiraterone Acetate).

In embodiments, devices and methods of this disclosure can provide viable, enriched cell sub-populations from heterogeneous tumors (biopsies, needle biopsies, body fluids etc.) for further analyses by off-chip studies to support personalized treatment decisions and efficacy control, i.e., genomic/mutational analyses, molecular and biochemical analyses for biomarker development, for drug screening, etc. to direct ongoing and further treatment.

In embodiments, the devices and methods can detect and enrich cancer stem or tumor-initiating cells by their bioelectrical fingerprint independent of specific genotype and can detect and enrich tumor cells from mixed biopsy or bodily fluid populations. Further, the devices and methods can detect and isolate different tumor stages for early detection and further analyses. Additional benefits of the devices and methods will be apparent to a skilled artisan.

Embodiments of the devices and methods can monitor the change of a cancer cell to more aggressive/less aggressive by their DEP response and monitor subsequent cultured cells to see if there is a shift in their drug response using DEP. As used in the context of this specification the terms DEP, cDEP, and iDEP may be used interchangeably. For example, a method specifying use of cDEP can alternatively or in addition employ the use of DEP and/or iDEP and vice versa. Further, the devices can operate in parallel or in series with other similar devices to enhance selectivity and throughput. Even further, a recirculation channel that allows for serial enrichment by multiple runs from the original sample volume can also be used. Further, embodiments also allow the methods to be run in reverse resulting in negative enrichment.

Embodiments of the present disclosure address the question of how can the physical properties of tumors, such as a cell's electrical, optical or mechanical properties, be used to provide earlier or more reliable cancer detection, diagnosis, prognosis, or monitoring of drug response or tumor recurrence.

To address patient health and monitoring, embodiments of this disclosure monitor changes in the electrical signatures of cells as they develop chemotherapeutic resistance or revert to a less aggressive stage. To address drug response and tumor recurrence, embodiments of this disclosure develop tools to isolate naturally occurring cells from tumor samples that exhibit, by exploiting differences in electrical properties.

To accomplish these tasks, embodiments of the present disclosure incorporate dielectrophoresis (DEP) and electrorotation (ROT)-based techniques, which manipulate cell motion based on their distinct bioelectrical signatures. Embodiments of methods of the present disclosure can use a novel method developed by the present inventors, contactless dielectrophoresis (cDEP). cDEP is ideally suited for in vitro studies because of the higher throughput compared to ROT or traditional DEP devices, and because it maintains the sterility of the sample in an isolate-and-culture platform, allowing for off-chip analysis. Using cDEP, the present inventors have shown that the bioelectrical signature of murine ovarian epithelial cells changes during cancer progression, allowing for a clear distinction of early (pre-malignant) and late stage cancer cells from resident peritoneal cells. Thus, embodiments of methods of the present disclosure utilize the electrical properties of tumors to identify the onset of drug-resistance and to isolate purified populations of chemotherapeutic resistant cells for in vitro analysis.

Dr. Davalos' team has invented a novel technique for cell characterization and separation known as contactless dielectrophoresis (cDEP) [20A, 21A] that overcomes conventional limitations by providing the spatially non-uniform electric field required for DEP while avoiding direct contact between electrodes and biological sample. This method maintains the sterility of the sample and allows for an isolate-and-culture platform or off-chip analysis. Dr. Roberts, in collaboration with Dr. Schmelz's group, has developed and characterized a syngeneic mouse cell model for progressive ovarian cancer [22A]. Mouse Ovarian Surface Epithelial (MOSE) cells of distinct phenotypes (morphology, growth rates, tumorigenicity) exhibit changes in their gene expression levels and cellular architecture also reported in the human disease [23A] that are associated with stage-specific changes in their biomechanical [24A] and bioelectrical properties [6A]. The availability of early (benign), transitional and aggressive stages in addition to stem-like or tumor-initiating (TIC) populations from all stages makes this a valid and innovative alternative to established human cell lines representing mostly cell-culture adapted late stages, allowing for the investigation of the impact of the specific tumor microenvironment on tumor growth and progression in immune competent mice; this is an important aspect in disease etiology but also critical for developing prevention and treatment strategies.

Aspect 1: Characterize the Bioelectrical Signature of Cells, for Example Cancer Cells, Associated with Drug Resistance.

Methods of the present disclosure can be used to determine the bioelectrical properties of different cells, such as drug-resistant and normal cells, using dielectrophoresis-based microfluidic devices. For example, cells can be derived from the inventors' syngeneic murine ovarian surface epithelial (MOSE) model. Samples of resistant and normal MOSE populations can be individually interrogated by DEP, such as cDEP and ROT, microdevices to quantify characteristic properties for each population.

Aspect 2: Validate the Bioelectrical Signature of Drug-Resistant Cancer Cells In Vivo for Comparative Assessment of their Tumor-Initiating Potential.

Cells harvested from in vivo derived solid tumors and ascites can be selectively sorted based on the bioelectrical signatures defined in Aspect 1, using DEP, such as cDEP, to isolate putative drug resistance populations. To achieve this end, specialized cDEP microdevices can be be designed via computational modeling to selectively separate drug-resistant from normal cancer cell populations. Sorting can be validated in vitro to verify the retention of drug resistance by quantifying response of sorted samples before and after exposure to the relevant chemotherapy. Finally, cDEP-identified cells can be implanted into the murine model and tumor outgrowth assessed for validation of the predicted resistance or tumor-initiating potential.

Impact:

The electrical signatures of drug-resistant and normal phenotypes from a syngeneic ovarian cancer cell line or human cell lines have not previously been determined. Embodiments of the present disclosure can enhance the fundamental understanding of relationships between drug-resistance and electrical characteristics, opening the door to rapid diagnosis and personalized treatment regimens at time of disease presentation. In addition, this may facilitate rapid monitoring of drug responsiveness and have prognostic value for predicting tumor recurrence rates.

Embodiments of methods of the present disclosure take advantage of the dielectrophoretic response of live cells to extract unique biophysical properties that allows for identification and differentiation of drug-resistant, tumor-initiating and drug-sensitive cancer cell types. This mapping of bioelectrical properties of chemotherapeutic-resistant cancer cells to disease stages of non-treated cells can allow for a new, rapid method for determining drug efficacy.

Methods of the present disclosure can be used to address one or more of the following: Acquisition of drug resistance and cancer stem-cell likeness or tumor-initiating capacity corresponds with unique bioelectrical signatures of cancer cells that can be utilized to 1) predict drug responsiveness or drug resistance and 2) to determine the cancer stem-cell composition and metastatic potential of solid tumors. Hence, the bioelectrical signature can serve as an important index of drug responsiveness or risk of tumor recurrence. According to embodiments, the inventors have demonstrated that tumor initiating cells can be isolated from normal tumor/cancer cells using DEP [37A]. Therefore, the technique for isolating cells based on drug resistance can also be applied to tumor initiating cells (cancer stem cells) and can be used to identify the optimal drug for treating a patient or to optimize a treatment regimen for a patient.

Additionally, methods of the present disclosure open the door for further development of a rapid biophysics-based method for predicting personalized patient response to chemotherapeutics. Mapping specific drug-resistance conditions to distinct bioelectrical signatures will translate to more reliable cancer diagnosis and drug-response monitoring.

Methods of the present disclosure are novel because, to the inventors' knowledge, the bioelectrical signatures of drug-resistant ovarian cancer cells independent of their genotype and their utilization for detection, treatment decisions and control have not been explored previously. In preferred embodiments, the present methods use contactless dielectrophoresis that in contrast to other dielectrophoretic methods support sterile high-throughput cell sorting and characterization, enabling sufficient cell samples for analysis. cDEP technology is rapid and amenable to mass production, making it viable for widespread use. Embodiments of this disclosure envision a form of personalized medicine in which a patient's peritoneal or solid tumor derived cells are assessed at time of presentation for their drug-resistant pre-inclinations in order to personalize their treatment regimen. The cell model utilized here is also novel, as it is a syngeneic mouse ovarian cancer line representing progressive ovarian cancer (not available as human model) that can be used to validate results in vivo in the specific tumor microenvironments.

Using embodiments of this disclosure, one can test whether bioelectrical properties of drug-resistant and non-resistant cancer cells differ by their dielectrophoretic responses, stemming from differences in cytoplasm ion content, nucleus-to-cytoplasm ratio, cytoskeleton structure, and membrane morphology and composition and other cell traits (size, morphology, internal organelles, etc.). These physical differences lead to distinctions in cytoplasm conductivity and specific membrane capacitance.

Methods of the present disclosure have the potential to define the specific bioelectrical fingerprint of drug resistant and stem-like ovarian cancer cells using the inventors well-characterized progressive mouse ovarian cancer cell system which now includes the tumor-initiating populations and/or the cancer stem-like populations that are derived from the different stages of cancer progression (early, intermediate and highly aggressive, poorly differentiated metastatic variants). The benchmarks for confirmation of their unique bioelectrical signatures will be based on their aggressiveness, their drug resistance/sensitivity profiles, tumor-initiating capacity, and metastatic potential: 1) Characterization of the specific bioelectrical fingerprint of drug resistant and stem-like ovarian cancer cells; 2) Bioelectrical profiling of cancer cells as they acquire drug resistance over time; 3) Correlation of the bioelectrical properties with in vivo and in vitro tumorigenicity and metastatic potential; 4) Validation of the results and translation to the human disease by comparison to established human cell lines that are drug responsive or resistant, and primary human cancer cells.

This defines a “bioelectrical signature” or “bioelectrical fingerprint” that identifies propensity towards drug resistance and allows for identification of personalized drug cocktail combinations for efficient treatment of cancer, such as advanced stage cancer.

One ultimate goal of embodiments of this disclosure is to use DEP, cDEP, or iDEP, to characterize ovarian cancer biophysics to determine the potential drug responsiveness of the individual patient at time of disease presentation. This provides a new focus for cancer treatment and drug-screening efforts from genotypic and biomarker-focused approaches with limited efficiency to characterization and sorting based on the biophysical properties of cancer cells. This will support effective treatment decisions and avoid the use of generic drug cocktails that may delay the use of individually targeted treatments. In addition, changes in the bioelectrical signature can be used for treatment control. The characterization of a specific stem-like bioelectrical fingerprint will allow for the immediate determination of the metastatic potential of the tumor and also direct treatment decisions. Together, these results could lead to the individual medicine approach that may prove to be more rapid and effective to enhance the survival of women with ovarian cancer than conventional treatment. The inventors approach is unique in that they can subject heterogeneous cell populations to bioelectrical profiling and effectively distinguish and sort unique subpopulations and validate biological functionality, drug resistance with in vivo tumorigenic potential. This new application will provide significant insight into the fundamental relationship between drug-resistance and stem-like properties. Embodiments also include a drug:cell profile library which will allow a quick test to determine an optimum regimen.

Approach

Aspect 1: Characterize the bioelectrical signature of cancer cells associated with drug resistance.

Here, the bioelectrical properties of cells can be experimentally determined using DEP-based microfluidic devices. The resulting profiles of resistant cells can be compared to those of TICs from the same line to determine whether resistance confers stem-like or tumor-initiating properties.

Methods of the Present Disclosure can be Used to Resolve Such Questions as:

1) Is the crossover frequency altered as cells acquire drug resistance? 2) Is the bioelectrical signature unique to a given drug? (Cisplatin/Carboplatin vs. a taxane such as paclitaxel or docetaxel vs. combinatorial resistance vs gemcitabine etc., also etoposide, and/or doxorubicin or vinorelbine. and 3) Can the cross-over frequency profile predict drug responsiveness prior to the onset of chemotherapy? Meaning can the inventors predetermine which combinatorial drug regimen will be efficacious prior to treatment?

Bioelectrical characterization of MOSE cells. The bioelectrical signature of cells can be determined by using DEP and electrorotation (ROT), label-free techniques for characterizing, detecting, and isolating cells based on their intrinsic biophysical properties [18A, 19A, 16A, 26A, 27A, 28A, 29A, 30A, 31A]. DEP is the motion of polarized particles in a non-uniform electric field toward the high (positive DEP) or low (negative DEP) gradients of electric field. ROT is the rotation of a cell or particle influenced by a rotating electric field [32A, 33A]. The time-average DEP force can be modeled as \vec{F}_{DEP}=2π∈ₘr³Re{K(ω)}∇(|\vec{E}ᵣₘₛ|²) where ∈ₘ is the permittivity of the suspending medium, r is the radius of the particle, \vec{E}ᵣₘₛ is the root mean square electric field, and Re[K(ω)] is the real part of the Clausius-Mossotti (CM) factor, k(ω) [34A]. The CM factor is given by (∈ₚ^{*}−∈ₘ^{*})/(∈ₚ^{*}−2∈ₘ^{*}), where ∈ₚ^{*} is the cell's complex electrical permittivity. K(ω) is a function of the frequency of the electric field, in addition to morphological and electrical properties of the cell. The crossover frequency, f_{co}, is the frequency at which the DEP force changes sign. In contrast to DEP force which depends on Re[K(ω)], ROT torque depends on the imaginary part, lm[K(ω)] and has its peak close to f_{co}.

Surface and membrane-related features such as cell size, shape, cytoskeleton, and membrane morphology affect the first crossover frequency and interior features such as cytoplasm conductivity, nuclear envelope permittivity, nucleus-cytoplasm volume ratio, and endoplasmic reticulum affect the second crossover frequency [10A]. The unique advantage of DEP and ROT is their ability to connect biophysical changes in cell structure to electrical properties. Thus, the K(ω) spectrum can be used as a tool to monitor the effect of treatments that physically alter the cell. Due to physical differences between drug-resistant and drug-sensitive cells, the inventors believe that they can select a distinct cell type based solely on biophysical properties by combining DEP and ROT.

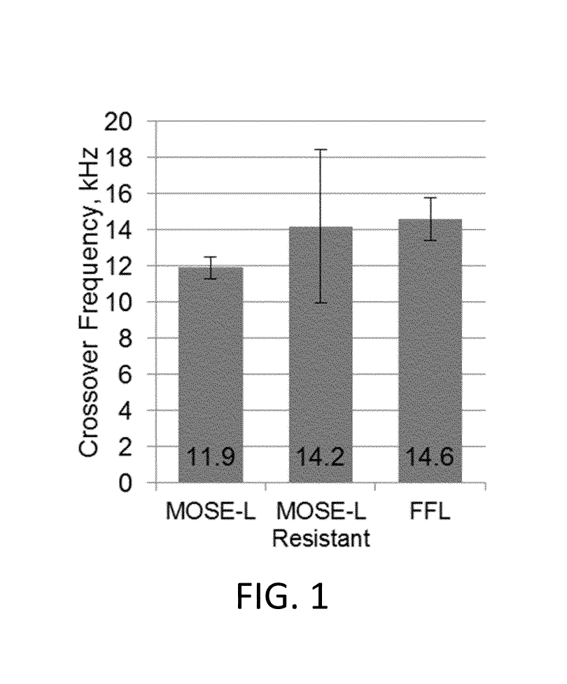

cDEP has been used in the inventors previous studies to demonstrate that MOSE cells can be differentiated by their bioelectrical crossover frequencies [6A], which were also distinctly different from stromal and immune cells [7A], indicating that cDEP can successfully be used to identify and differentiate ovarian cancer cells from peritoneal cells. In a chemotherapeutic approach, the inventors demonstrated that sphingolipid metabolites with anti-tumor properties or pro-tumorigenic activities differentially modulate the bioelectrical characteristics of late stage MOSE (MOSE-L) cells towards a profile similar to that of benign MOSE E cells. This was associated with a shift of the specific membrane capacitance of MOSE-L cells towards that of MOSE-E cells [8A]. Since preliminary studies showed that cisplatin and paclitaxel treatment altered cytoskeleton organization and surface topography (Schmelz, unpublished observations), the inventors examined the DEP profiles of a cisplatin-resistant variant derived from MOSE-L cells, the MOSE-LcisR as well as a highly aggressive TIC variant that expresses the reporter gene firefly luciferase, MOSE-L/FFLTICv. The latter cell variant was selected by culture in stem cell-like, spheroid conditions in serum-free media and represented only a minor fraction of the total cell population of the MOSE-L parental line (ca. 0.001%). These cells are able to clonally expand from single cells in serum-free conditions and as few as 100 cells are sufficient to induce widespread peritoneal tumor dissemination in immune competent, syngeneic C57BL/6 female mice (Roberts, personal communication). As depicted in FIG. 1, both cell lines display unique crossover frequencies that allow for their identification and differentiation from the MOSE-L parental cells. Although preliminary, these data strongly support that the crossover frequency can be employed to detect and characterize cancer cells based on aggressiveness as well as drug responsiveness and tumor-initiating capacity [37A].

Establishment of Drug-resistance and Determination of the Drug-specific Bioelectrical Signature. The unique bioelectrical signatures of drug resistant variant cell lines (e.g., derived from MOSE-L cells and their TICs) can be determined and whether these are unique for a given drug, or any combination thereof. Here, a comprehensive profiling approach is provided comparing the bioelectrical crossover frequencies of common chemotherapeutic drugs: cisplatin/carboplatin, paclitaxel, docetaxel, either alone and in combination (both compound groups are used in standard chemotherapy), vs gemcitabine, etoposide, and daunorubicin/adriamycin or vinca alkaloids such as vincristine, vinorelbine or combinations thereof as representatives for commonly used compounds in standard ovarian cancer care, and experimental chemotherapeutics. Similar to establishment of the inventors MOSE-LcisR variant (IC50>2 uM vs IC50<0.25 uM for MOSE-L parental cells), additional drug resistant variants can be established using standard cell culture passaging of MOSE-L, in the presence of step-wise increasing doses of chemotherapeutic agents. Importantly, one can be assess the bioelectrical signature as a function of time post-treatment. Thus, as cells progressively acquire drug resistance the direct correlation with dosing and modulation of bioelectrical signature can be determined. This will allow for further refining of the bioelectrical parameters as well as confirm whether there is an early bioelectrical signature that predicts emergence of drug resistance. This may prove useful in identifying subpopulations of cancer cells that display a propensity for emergence of drug resistant variants.

FIG. 1 is graph showing crossover frequency for MOSE cells. The FFL stem cell electrical signature increases compared to the MOSE-L parent stage cells.

The bioelectrical signatures of three human ovarian cancer cell lines, A2780 (derived from solid tumor tissue), SKOV3 and OV90 (both derived from malignant ascites) as they acquire drug resistance over time can be compared. In some cases, the uniqueness of the drug-specific bioelectrical signature can be confirmed and allow for the validation of signatures obtained with the MOSE-L drug resistant variants, which may provide invaluable insights towards a personalized screening approach to optimize which drug cocktail may be most beneficial for a given patient. It is believed that the acquisition of drug resistance confers cancer stem-like or tumor-initiating properties to the cells. In some cases, the bioelectrical signature can be used to define the TIC population of cells derived from different stages of neoplastic progression as a metric for metastatic potential and as such can provide insight into the fundamental relationship between drug-resistance and stem-like properties.

Microdevice fabrication. For cDEP, a silicon master stamp can be fabricated on a silicon substrate using Deep Reactive Ion Etching (DRIE) process. The scalloping effect, sidewall roughness due to the DRIE etching method, can be removed by a 5 min wet etching using TMAH 25% at 70° C. The liquid phase PDMS, made by mixing the monomers and the curing agent in a 10:1 ratio (Sylgrad 184, Dow Corning), can be poured onto the silicon master and can cure for 45 min at 100° C. The cured PDMS can be removed and fluidic connections can be punched through with blunt needles. The PDMS replica can be bonded to clean glass slides after treating with air plasma for 2 min. These devices lend themselves to mass fabrication as well through either extrusion or stamping based methods with suitable materials. The lab has also experimented with CO₂ laser etched polymers with multi material membranes. ROT devices can be based on quadripolar electrodes with gold electrodes deposited on glass using Pressure Vapor Deposition and a titanium seed layer [38A]. These can also make these as cDEP devices or fluid electrodes.

Apparatus. AC fields can be applied to the microdevices using a combination of a function generator a wideband power amplifier and HV transformer to provide voltage needed. A syringe pump can used to drive samples through the microfluidic chips. An inverted or other style microscope (DMI 6000B, Leica) including digital media capture devices or those with specialized filters attached can be used for monitoring cells in the main channel and the dielectrophoretic behavior of the cells can be recorded. All electrical devices (function generator, oscilloscope, syringe pump, microscope) can be connected to single computer allowing for control and data acquisition. Microdevices can be placed in a vacuum jar for at least 30 min before running the experiments to reduce priming issues. The electrode channels and main microchannel can be primed with PBS and then filled with respective fluids: high conductivity electrode and cell suspension mixture.

Determination of DEP signature. The inventors can measure the crossover frequencies and the ROT spectra using the devices described above, mounted on an inverted microscope equipped with a camera for recording videos of cell motion in response to the field. Cells can be suspended in a low conductivity solution [39A] and placed in the center of the electrodes. For cDEP, the AC signal can be programmed to sweep across frequencies in sync with video recording. For ROT, AC fields with 90° offsets can be generated by in-house electronics utilizing a THS3092 board, ultimately capable of supplying 7.5 Vpp at 50 Hz-5 MHz. MATLAB can be used for image processing of the video from each experiment. From cDEP experiments the CM can be determined as the frequency at which cells experience no force. For electrorotation experiments the inventors can use a MATLAB script to determine when maximal angular velocity occurs. The inventors can fit CM theoretical curves to all measured data and determine changes in bioelectrical properties of cells.

Imaging cells to validate biophysical properties. SEM or other microscopy such as confocal microscopy can be used to observe the surface roughness, and immune-fluorescence or confocal microscopy to determine the cytoskeletal structure and membrane morphology.

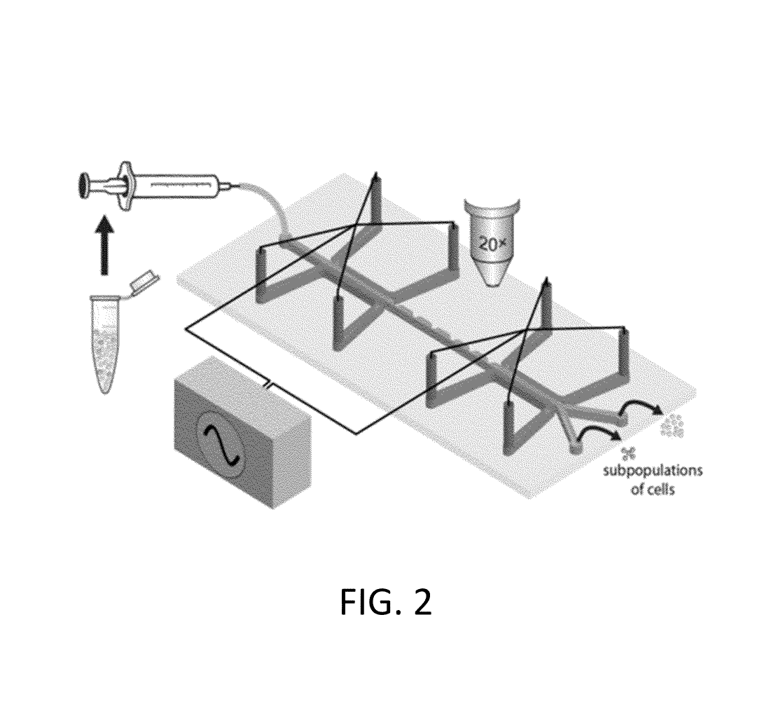

FIG. 2 is a schematic diagram showing a cDEP Device Schematic.

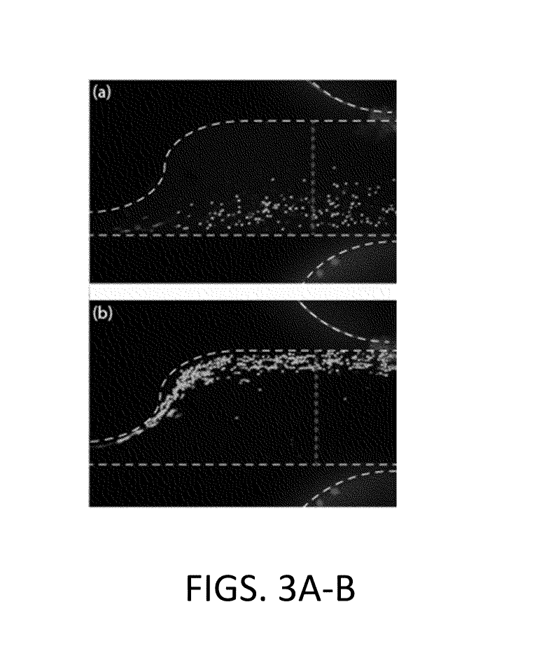

FIGS. 3A and 3B show MOSE cells experiencing negative and positive DEP, respectively.

Statistical Analysis: Statistical analysis can be used to determine if the gathered data of different cells are significantly different.

Potential pitfalls and alternative approaches. There may not be a unique drug specific bioelectrical signature and all drug resistant variants irrespective of the drug may exhibit the same unique global bioelectrical profile based on crossover frequency. Based on a recent report on impedance changes with drug resistant breast cancer cells [40A] and the inventors' results, this is highly unlikely. Measuring DEP response on a wider frequency range that includes the second crossover frequency, measured by ROT experiments, compared to limiting measurements near the first fco, should provide more sensitive profiling.

Embodiment: fco can be measured and it can be determined how biophysical parameters (e.g., cytoplasm conductivity, membrane permittivity) of cells resistant to chemotherapeutics differ from those of normal cancer cells.

Using embodiments of the invention, the bioelectrical signature of drug-resistant cancer cells in vivo for comparative assessment of their tumor-initiating potential can be validated. In some embodiments, the bioelectrical signature may or may not be maintained during in vivo dissemination of cancer. More specifically, in some circumstances, ascites-associated cancer cells differ from solid tumor associated cancer cells with respect to their bioelectrical signature. According to embodiments, an objective is to employ bioelectrical profiling to predict drug responsiveness at time of presentation.

Prior to in vivo assessment of the bioelectrical signature of tumor cells, the profiling and sorting protocol, using heterogeneous populations of cells, including the different drug resistant variants, TICs, and fully differentiated parental cells can be used. Cell sorting can be performed at frequency and voltage where subpopulations of cells differ the most. In continuous sorting mode, mixed cell suspensions can be pumped in the channel, focused to narrow stream, and deflected towards appropriate outlets by DEP force. In batch sorting mode, cells can be pumped in the channel and one type of cells can be trapped in the regions of high electric field, while other cells can continue to outlet. Untrapped cells can be collected from outlet, trapped cells can be then released from the channel, and collected. The sorted and enriched populations can be subsequently validated for their putative phenotypes as described in more detail below. Hence, putative drug-sensitive populations should still exhibit sensitivity to all or selective drugs, the putative drug-resistant subsets can be verified by the retention of their drug resistance profile (IC50 assays), and the putative TICs can be confirmed by their enhanced capacity for clonal growth under stem-like growth conditions in serum-free media (spheroid assay) and further defined by their TIC frequency (aka: c-SCF) as determined by extreme limiting dilution analysis [41A]. The putative non-TIC/drug-sensitive subset populations can be used as a negative control for validation of c-SCF. According to embodiments, it can be determined whether the drug resistant phenotype and the TIC populations are identical or unique with respect to bioelectrical properties. In some circumstances, the TIC population may represent a unique subset population residing within each drug resistant population.

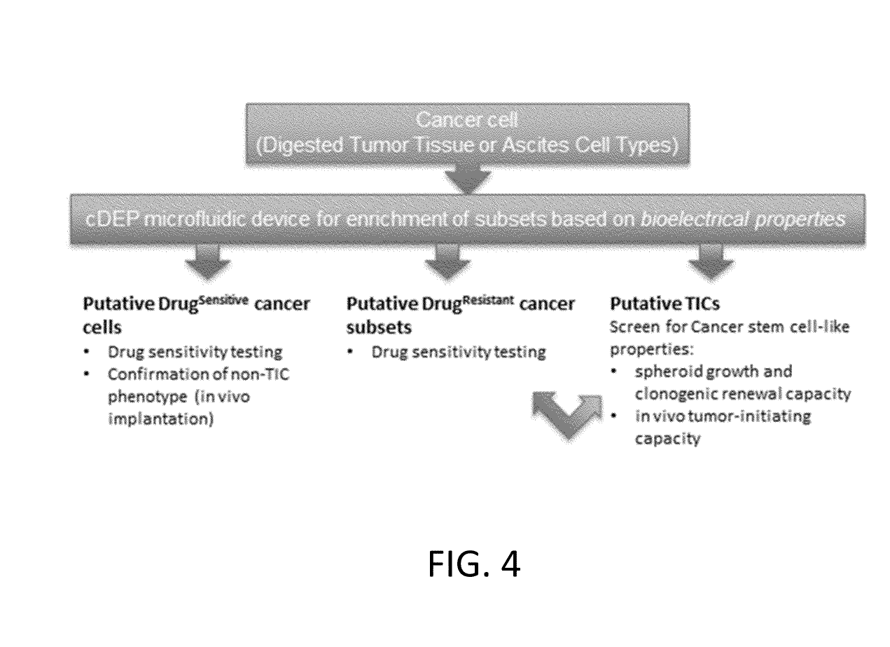

Analysis of cells from harvested tumor tissue. One validation step can include to confirm that the in vitro culture conditions are not inadvertently impacting the bioelectrical signatures of cells and determine the impact of the specific tumor microenvironment on signatures. Therefore, tumor tissue harvested from syngeneic C57BL/6 mice intraperitoneally implanted with 1×104 MOSE-L/FFL-EGFPTICv cells (n=10) can be harvested post-mortem, digested and subjected to bioelectrical profiling of the tumor-associated cells. This dose results in widespread tumor formation throughout the peritoneal cavity, including the omentum, peritoneal lining, diaphragm, liver, and visceral fat depots; ascites is typically observed in 60% of the mice. Tissue and peritoneal serous fluid harvest can be performed as previously described by the Roberts and Schmelz laboratories [42A]. Briefly, collected ascites cells and tumor tissue (omentum, diaphragm, mesentery) can be disrupted by mild collagenase/hyaluronidase digestion and filtered through 80 and 20 μm cell strainers to obtain single cell suspensions prior to bioelectrical profiling. Cell suspensions can be characterized with cDEP devices in similar fashion. As the MOSE-L/FFL-EGFPTICv represents a tumor-initiating variant, the inventors fully anticipate that upon in vitro propagation, both TIC and more differentiated non-TIC phenotypes can be evident and realized by cDEP. In fact, if the drug-resistant phenotype is already identifiable in this cell line, then an enrichment of the drug-resistant fingerprint is also anticipated. The inventors expect to be able to isolate and differentiate between these subsets based on the voltage and frequency of the applied field. In a similar fashion, the inventors can implant a heterogeneous suspension of drug sensitive and resistant cells and following tumor outgrowth validate bioelectrical profilings' ability to differentiate between subsets of drug resistant cells. A summary of these experiments is shown in FIG. 4.

Device optimization by computational modeling. The spatial gradients of the electric field are dictated by the channel geometries, and can be predicted by numerical modeling. The electric field gradient distribution and fluid dynamics can be modeled computationally using Comsol 4.2 (Burlington, Mass.). The inventors have shown [43A, 44A, 45A] that determination of the field distribution within the channel and the predicted cell trajectories enable computational optimization of device designs. Results were obtained using a device with sawtooth-shaped constrictions that operates near the first crossover frequency of cells where they are more easily separable due to distinct CM factor spectra. The inventors can further optimize this device to reach the high level of sensitivity and selectivity required to select drug-resistant and/or tumor-initiating cells from a heterogeneous suspension of tumor-associated cells by iteratively re-designing and computationally modeling constrictions in the sample channel in order to have the same field gradient across the whole cross-section of the channel such that cells can be exposed to the same amount of DEP force. The inventors expect that this can increase separation resolution. For increased resolution the investigators can choose to use an number of microfluidic techniques detailed in literature including hydrodynamic focusing. Alternatively the inventors can focus the initial stream of cell suspension by DEP with another pair of electrodes which causes negative DEP to all cells. For multi-frequency devices, the inventors can build electronics capable of independently generating two HV sine signals and design new cDEP devices featuring multiple electrode channels in succession or at divergent axes. The inventors can optimize shape of the channels and throughput to ensure sufficient viable cells out of the device for spheroid formation and in vivo studies. This would yield cDEP devices which employ a multi-frequency cascading platform with multiple outlets to search for subpopulations of resistant cells.

In cases where cells may not differ enough to separate them at one frequency and amplitude, a multi-frequency strategy can be employed for conventional DEP cell separation [46A]. For example, cells can be separated in sequential steps, such as two or more sequential steps, each operating at two simultaneous frequencies to assess whether there are subpopulations of resistant cells. The heterogeneity of mouse-derived cells may perturb characterization of subpopulations of the cancer cells. In such cases, the cells can be separated with cDEP devices prior to characterization and then the bioelectrical signature of the separated cells can be measured.

Embodiments of this disclosure provide a dielectrophoresis device having a sample channel which is separated by physical barriers from electrode channels which receive electrodes. The electrodes provide an electric current to the electrode channels, which creates a non-uniform electric field in the sample channel, allowing for the separation and isolation of particles in the sample. As the electrodes are not in contact with the sample, electrode fouling is avoided and sample integrity is better maintained.

Additional embodiments of this disclosure provide a dielectrophoresis device having a sample channel which is separated by physical barriers from electrode channels which receive electrodes, whereby the sample channel and electrode channels are formed in a single substrate layer and whereby the physical barriers are formed by the substrate itself.

Additional embodiments of this disclosure provide a dielectrophoresis device having a channel for receiving a sample in a first substrate layer, a first electrode channel and a second electrode channel for receiving electrodes in a second substrate layer and an insulation barrier between the first substrate layer and the second substrate layer.

Additional embodiments of this disclosure provide a dielectrophoresis device having a first electrode channel for conducting an electric current in a first substrate layer, a channel for receiving a sample in a second substrate layer and a second electrode channel for conducting an electric current in a third substrate layer. The device also has a first insulation barrier between the first substrate layer and the second substrate layer and a second insulation barrier between the second substrate layer and the third substrate layer, preventing the sample from coming in contact with the electrodes.

### EXAMPLE

Ovarian cancer, the most frequent cause of death from gynecological malignancies in women and the fifth leading cause of death from cancer in women [1, 2] is a genetically and histologically heterogeneous disease. The lack of common genetic markers hinders both cancer detection at earlier stages and the development of successful treatment options.

Development of treatment regimens and detection techniques that do not rely upon the expression of specific genes or surface markers could ameliorate these challenges. The operating principle for the cell manipulation and characterization strategy disclosed in this specification is dielectrophoresis (DEP), the movement of polarized particles in a non-uniform electric field [3]. DEP can be applied as a cell manipulation technique [4-7] that does not rely on genotype-dependent biomarkers, in contrast to other cell isolation techniques such as flow cytometry [8] and magnetic bead cell separation [9]. DEP has been successfully used for drug screening applications [10] to distinguish between multidrug-resistant and sensitive cancer cells by their cytoplasmic conductivity [11,12], and to determine cytoplasm and membrane conductivity of drug-treated red blood cells [13]. Further applications of DEP include cell viability determination [10,14] and investigations of drug-stimulated cell surface roughness increase [15]. In conventional DEP techniques, metallic electrodes are used to create a nonuniform electric field [10-15]. However, contact between electrodes and the sample fluid can in some circumstances create challenges for manipulating biological samples including Joule heating, sample contamination, and bubble formation due to electrolysis. To address these issues, a contactless DEP (cDEP) can be used, which is a microfluidic cell manipulation strategy that eliminates direct contact between electrodes and the sample [16]. In cDEP, an electric field is generated using electrode channels that are separated from the sample channels by a thin insulating barrier. These electrode channels are filled with a highly conductive fluid and under an alternating current (AC) signal are capacitively coupled to the sample channel [17-20].

cDEP has been used to isolate prostate tumor initiating cells from prostate cancer cells [21] cancer cells from blood cells [22, 23] viable from dead cells [17] and different stages of breast cancer cell lines [24]. Moreover, cDEP has previously been used to quantify dielectric properties of a syngeneic mouse cell model for progressive ovarian cancer [25]. In this model, isolated primary mouse ovarian surface epithelial (MOSE) cells undergo transformation in vitro and progress to malignant stages [26]. Since human cell lines providing different stages of ovarian cancer derived from one genetic source are not available for study, the MOSE model represents a useful alternative that avoids the potential confounding variable of inter-subject genetic differences. Based on their phenotype, MOSE cells were categorized into early, intermediate, and late stages of malignancy. An increasingly dysregulated cytoskeleton organization and changes in the expression of cytoskeleton genes and their regulators were observed during neoplastic progression, accompanied by an increase in membrane ruffles and protrusions [26, 27]. Cytoskeletal changes were associated with stage-specific changes in cellular biomechanical properties [28]. Also, the inventors have recently shown for the first time that the dielectric responses of cells are different in different stages of progression [25]. The inventors compared the crossover frequency and membrane capacitance of different stages of MOSE cells, finding that the membrane capacitance was greater in malignant cells compared to benign cells [25]. Aggressive MOSE cells also showed different dielectric responses from peritoneal cells, specifically macrophages and fibroblasts [29] indicating that cDEP may be an option for isolating ovarian cells from peritoneal fluid for cancer detection.

Current cancer treatments rely upon highly toxic doses of chemotherapeutics and can cause severe adverse side effects. In addition to achieving early detection, the development of less aggressive treatment options that at least partially reverse the aggressive phenotype of the disease to an earlier, more benign state and therefore may turn a deadly cancer into a chronic disease could be highly beneficial for patients. In this regard, the inventors have used orally administered complex sphingolipids to successfully suppress colon and breast cancer [30-34].

Sphingolipid metabolites influence membrane biology and as lipid second messengers modulate cellular homeostasis, functions and responses to extracellular stimuli. Sphingolipids are involved in the regulation of cell growth, cell death, migration, angiogenesis, and metabolism, among many other cell functions [35, 36]. Dysregulation in metabolic pathways of sphingolipids can cause progression of some diseases, including cancer [37, 38]. The sphingolipid metabolites ceramide (Cer), sphingosine (So), and sphingosine-1-phosphate (S1P), can stimulate opposing cellular responses depending upon their relative levels in a cell, forming the so-called sphingolipid rheostat [39, 40]. In general, So and Cer are known as cell death-promoting factors leading to apoptosis, inhibition of cell growth, differentiation, migration, and angiogenesis [41] and thus could be considered tumor suppressors. However, Cer has also been associated with inflammation [42] suggesting a tumor promoting effect. In contrast, S1P acts to support growth and survival of numerous cell types. As such, it has tumor-promoting effects, including inhibition of apoptosis and stimulation of angiogenesis, cell proliferation, differentiation, and migration [41, 39]. Elevated levels of S1P have been reported in human ascites fluid of patients with ovarian cancer [43] and may promote the survival, adherence, and outgrowth of peritoneal metastases.

Interestingly, therapies targeting S1P generation and signaling have led to a decreased tumor formation in mice [44]. The inventors have used cDEP to characterize MOSE cells' electrical properties after So and S1P treatment to compare the effects of exogenous sphingolipid metabolites associated with anti- and pro-cancer effects, respectively. The inventors demonstrate that sphingolipid modulation therapy induced distinct changes in the bioelectrical properties of cancer cells.

Importantly, the treatments were non-toxic, allowing the use of cDEP to discriminate among viable MOSE-derived cancer cells. The inventors report that So treatment correlated with a shift in electrical properties of the aggressive MOSE cells towards a profile reminiscent of more benign stages, whereas S1P did not significantly impact the electrical properties of either early or late stage MOSE cells. The association of the altered electrical phenotype of the So treated cells with cancer suppression and the potential for use of the electrical phenotype as a marker for treatment efficacy can be explored in future studies.

Theory

While the inventors do not wish to be bound to any particular theory, it is believed that a particle located within the boundaries of an applied nonuniform electric field will become polarized and experience a dielectrophoretic force, described by:

\vec{F}_{DEP}=(p_{eff}·∇)\vec{E}_{RMS}, (1)

p_{eff} is the effective induced dipole moment of the particle and \vec{E}_{RMS} is the root mean square electric field. For a lossy spherical particle where:

p_{eff}=4π∈ₘr³Re[K(ω)]\vec{E}_{RMS}, (2)

and ∈ₘ is the permittivity of the suspending medium, r is the radius of the particle, and the Clausius-Mossotti factor is represented as:

K(ω)=(∈ₚ^{*}−∈ₘ^{*})/(∈ₚ^{*}+2∈ₘ^{*}), (3)

The real part of the Clausius-Mossotti factor is theoretically bound by −0.5 and 1, and ∈ₚ^{*} and ∈ₘ^{*} represent the complex permittivity of the particle and the suspending medium, respectively, where the complex permittivity is ∈*=∈+σ/jω. The sign of the frequency dependent Clausius-Mossotti factor determines the direction of translational particle movement, either toward a region of high electric field gradient (positive DEP, pDEP) or low electric field gradient (negative DEP, nDEP). Biological particles are more complex than a simple spherical particle, and models of varying complexity have emerged that can approximate a biological particle, such as a cell, with sufficient accuracy. In a multi-shell model [45] the membrane of the bioparticles, the nucleus, and even the nucleus membrane can be considered, and parameters can be tailored to a specific cell of interest. For the work presented here, a single shell model that considers the cell's thin lipid membrane and the internal cytoplasm is used. Thus the effective permittivity can be written as:

$${ɛ}_{p}^{*} = {ɛ}_{2}^{*} \frac{{γ}^{3} + 2 ( \frac{{ɛ}_{cyt}^{*} - {ɛ}_{mem}^{*}}{{ɛ}_{cyt}^{*} + 2 {ɛ}_{mem}^{*}} )}{{γ}^{3} - ( \frac{{ɛ}_{cyt}^{*} - {ɛ}_{mem}^{*}}{{ɛ}_{cyt}^{*} + 2 {ɛ}_{mem}^{*}} )} \qquad ( 4 )$$

where γ³=r/(r−d), d is the thickness of the membrane, r>>d, and ∈_{cyt}^{*} and ∈ₘₑₘ^{*} are the cytoplasm and membrane complex permittivity, respectively.

For each cell type, within a specific media, there exists a unique crossover frequency, fₓₒ. At this frequency the real part of f_{CM} equals zero, thus, there is no net DEP force acting on the cells. The first crossover frequency of mammalian cells in low conductivity buffer of 100 μS/cm [46] occurs between 10-100 kHz, and the second crossover frequency is typically on the order of 10 MHz. Cell size, shape, cytoskeleton, and membrane morphology affect the first crossover frequency, while cytoplasm conductivity, nuclear envelope permittivity, nucleus cytoplasm (N/C) volume ratio, and endoplasmic reticulum influence the second crossover frequency [47]. Thus, the crossover frequency can be used as a tool to monitor the effect of treatments that physically alter the cell. The crossover frequency can be determined by setting Re{K(ω)} equal to zero and solving for frequency. Then, fₓₒ is found by:

$${f}_{xo} = \frac{1}{2 π} \sqrt{\frac{( {σ}_{p} - {σ}_{m} ) ( {σ}_{p} + 2 {σ}_{m} )}{( {ɛ}_{p} - {ɛ}_{m} ) ( {ɛ}_{p} + 2 {ɛ}_{m} )}} . \qquad ( 5 )$$

For frequencies less than 1 MHz, dielectric properties of cells are related to membrane properties [48]. The specific capacitance of the cell membrane, Cₘₑₘ, and conductance associated with the transport of ions across the membrane, Gₘₑₘ can be defined as:

$$\begin{matrix}{C}_{mem} = \frac{{ɛ}_{mem}}{d} , & ( 6 ) \\ {G}_{mem} = \frac{{σ}_{mem}}{d} , & ( 7 )\end{matrix}$$

and the total effective conductance per unit area of the cell membrane, as Gₘₑₘ^{*} [46] as

$${G}_{mem}^{*} = \frac{2 {K}_{ms}}{{r}^{2}} + {G}_{mem} , \qquad ( 8 )$$

Kₘₛ is the surface conductance of the membrane related to the electrical double layer around the cell, and Gₘₑₘ is the conductance associated with the transport of ions across the membrane [46, 49]. At low frequencies, <100 kHz, the low value of Gₘₑₘ, representing the membrane bulk conductivity, prevents the applied electric field from penetrating the interior of the cell. As the frequency increases beyond 100 kHz, membrane resistance begins to short-circuit and electric field penetrates inside the cell. Then, for frequencies below 100 kHz Equation (5) can be simplified to the form of [46]:

$${f}_{xo} = \frac{\sqrt{2} {σ}_{m}}{2 π r {C}_{mem}} - \frac{\sqrt{2} {G}_{mem}^{*}}{8 π {C}_{mem}} \qquad ( 9 )$$

The second term on the right hand side can be neglected for Gₘₑₘ^{*}<<4σₘ/r. This inequality is valid for low conductivity media, such as the cell solution in this work (conductivity of approximately 100 mS/m). Thus, the second term is negligible and the crossover frequency can be calculated from:

$${f}_{xo} = \frac{\sqrt{2} {σ}_{m}}{2 π {rC}_{mem}} \qquad ( 10 )$$

Equation (10) shows that there is an inverse relation between the ratio of crossover frequency to sample conductivity, fₓₒ/σₘ and Cₘₑₘ. Also it shows that the electrical properties of cells, such as specific membrane capacitance, can be calculated from their crossover frequency.

Although the single-shell model has been successful for predicting the biophysical properties of cells, it sometimes deviates from the experimental results [50] since the real cellular structure is more complex than that assumed by the single-shell model. For instance, this model assumes cells have a thin and spherical membrane which surrounds a spherical homogeneous interior, and thus does not take into account membrane inhomogeneity and cytoplasm and nuclear structural features [51]. Consequently, this model cannot correlate specific membrane capacitance, Cₘₑₘ, with membrane morphological complexity.

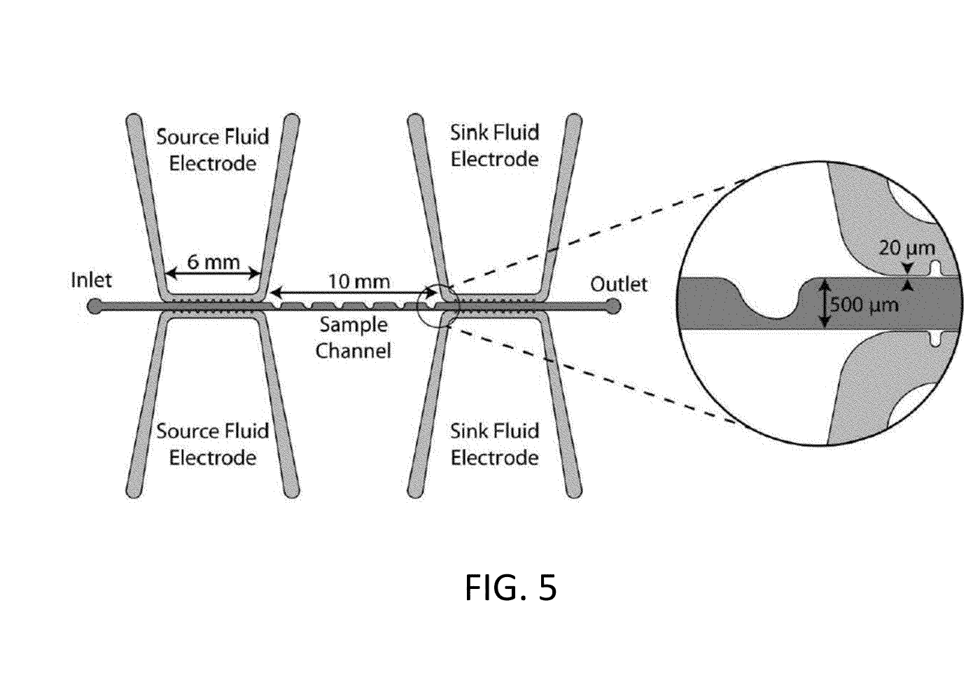

The microdevice, shown in FIG. 5, consists of a straight main channel and parallel fluid electrode channels, each 50 μm in depth. Other devices can also be used including those disclosed in U.S. Pat. No. 7,678,256 and U.S. Application Publication Nos. 2010/0224493 and 2012/0085649. In this embodiment, the main channel has an inlet and outlet with a series of rounded ‘sawtooth’ features that constrict the main channel from 500 μm width to 100 μm. These sawtooth features create high electric field gradients in the region where the sample channel is constricted, and the series of features increases the length of time that the cells are exposed to the DEP force. Fluidic electrode channels are separated from the sample channel by 20 μm thick insulating barriers. Throughout this specification, the side of the channel which has sawtooth features will be referred to as top side of the channel and the opposite side of the channel which is a straight wall will be referred to as bottom side of the channel.

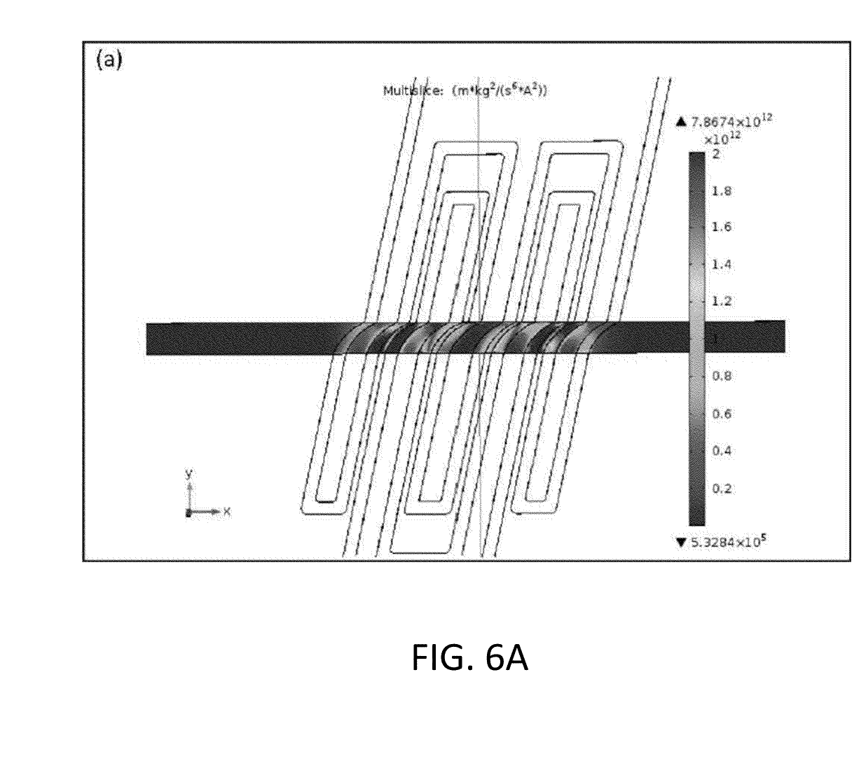

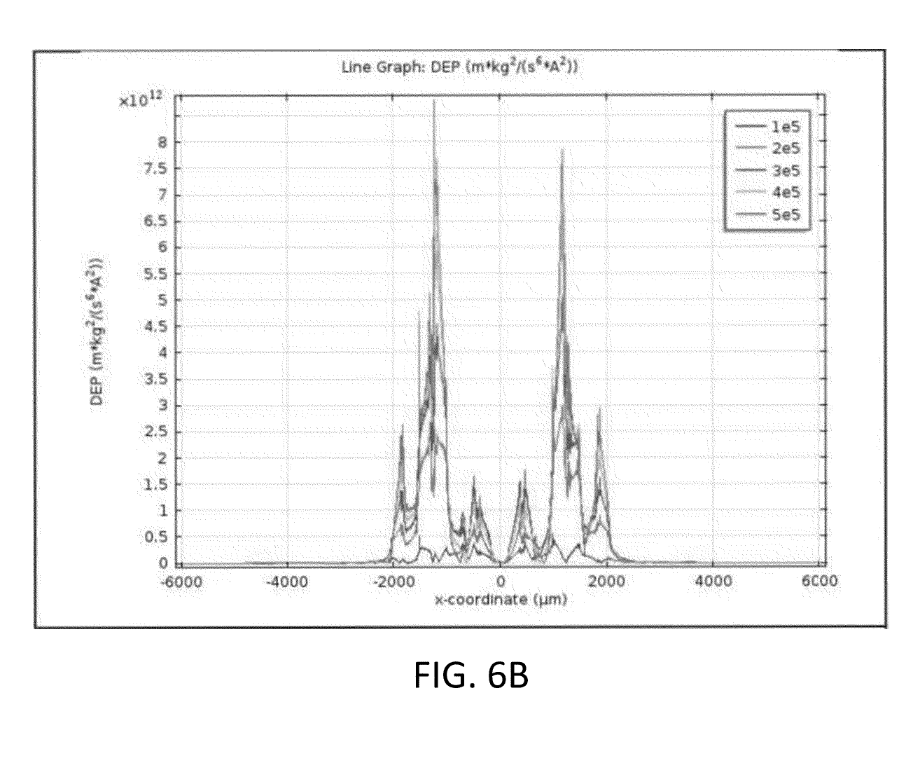

For example, microdevices can consist of a straight main channel and overlaid interdigitated fluid electrode channels. The main channel has an inlet and outlet and the height and width can be tailored for the desired throughput or application. The overlaid electrode channels consist of curved geometry. These channels are separated from the main channel by a 20 ˜m thick insulating layer. The curves taper from the bottom side of the channel to the top side (FIG. 6A, 7A). This geometry can be altered to achieve a desired DEP force given a particular frequency by changing parameters such as the angle of curvature, nominal ratio of electrode width to gap width, and number of electrode pairs in series. The nominal ratio of electrode width to gap width is determined by the distances at the bottom of the channel.

FIG. 6A shows a surface plot for computational modeling of ∇(\vec{E}_{RMS}·\vec{E}_{RMS}) in the sample channel of a multilayer cDEP device with over laid curved tapered electrode channels. The plot was generated at 100 V, 200 kHz. There are 4 electrode pairs, the included angle of the curves is 45′, and the nominal ratio of electrode channel width to gap width is 2:1. FIG. 6B shows a plot along x-coordinates at z=35 y=0 of the change in ∇(\vec{E}_{RMS}·\vec{E}_{RMS}) with x-position and frequency. The legend (units of Hz) shows the frequency range 100-500 kHz.

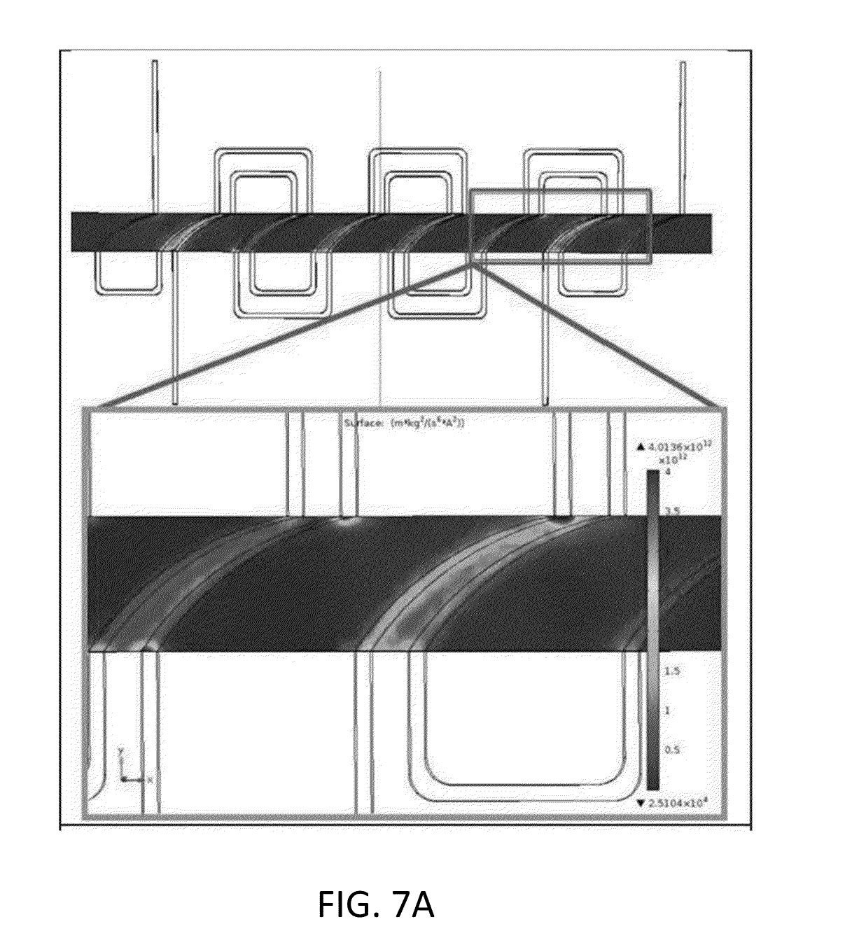

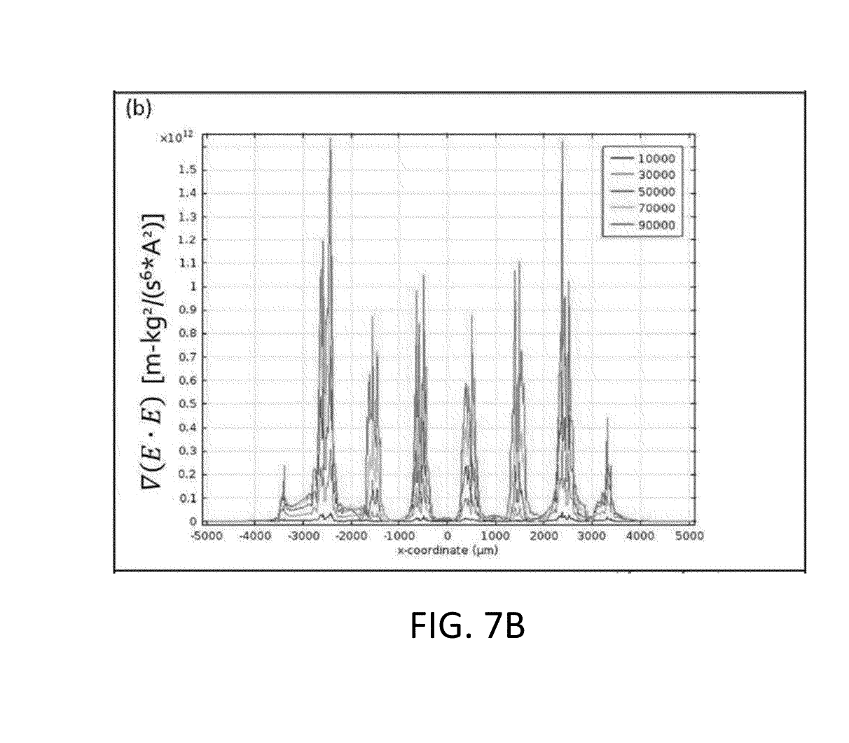

FIG. 7A shows a surface plot for computational modeling of ∇(\vec{E}_{RMS}·\vec{E}_{RMS}) in the sample channel of a multilayer cOEP device with curved tapered electrode channels. FIG. 7B shows a line plot along the x-centerline of the sample channel demonstrating the change in ∇(\vec{E}_{RMS}·\vec{E}_{RMS}) with frequency. The legend shows the frequency value in Hz.

Device Fabrication

A stamp of the microdevice design was made for the use with standard soft lithography techniques. AZ 9260 photoresist (AZ Electronic Materials, Somerville, N.J., USA) was spun onto a clean silicon wafer and exposed to UV light for 60 s through a mask patterned with the device design. The exposed photoresist was removed using AZ 400 K developer (AZ Electronic Materials, Somerville, N.J., USA). Deep Reactive Ion Etching (DRIE) was used to etch microchannels to a depth of 50 μm. Surface roughness on the side walls was removed by 5 minutes wet etching with tetramethylammonium hydroxide (TMAH) 25% at 70° C. A thin coating of Teflon, which improved the release of the device from the stamp, was deposited using DRIE.

The devices were fabricated from polydimethylsiloxane (PDMS). PDMS was mixed in a 10:1 ratio of elastomer to curing agent (Sylgard 184, Dow Corning, USA). The liquid-phase PDMS was left under vacuum for 30 minutes to remove air bubbles, and was then poured onto the silicon master stamp and cured for 45 minutes at 100° C. Upon removal from the wafer, the device was trimmed and fluidic connections were punched in the inlet and outlet of each channel with a 1.5 mm blunt puncher (Howard Electronic Instruments, USA). The PDMS device and a glass microscope slide were cleaned before treating with air plasma for two minutes and bonding together.

Cell Culture and Drug Treatment

MOSE cells were cultured in high glucose DMEM (Sigma Aldrich) supplemented with 4% fetal bovine serum (Atlanta Biologicals), 3.7 g/L NaHCO₃, and 1% penicillin/streptomycin (Sigma Aldrich). MOSE-E and MOSE-L cells were treated with 1.5 μM So or 500 nM S1P as BSA complexes (BSA, fatty acids-free fraction V, Calbiochem) for three passages, allowing 3-4 days between each passage. These treatments were not toxic to the cells.

Cell Preparation

The cells were harvested by trypsinization, washed and resuspended in DEP buffer (8.5% sucrose [wt/vol], 0.3% glucose [wt/vol], 0.725% RPMI [wt/vol]) [52] to a concentration of 3×106 cells/mL. The cells were stained with Calcein-AM (Molecular Probes Inc., Carlsbad, Calif., USA), at a concentration of 2 μL dye per mL cell suspension. The final cell suspension had an averaged conductivity of 96.97±4.15 μS/cm, measured using a conductivity meter (Horiba B-173 Twin Conductivity/Salinity Pocket Testers, Cole-Parmer).

Experimental Setup

The PDMS device was placed under vacuum for 30 minutes immediately prior to priming the main channel with the cell suspension. The cell suspension was introduced to the main channel inlet through Teflon tubing attached to a syringe with a needle tip (Cole-Parmer Instrument Co., Vernon Hills, Ill.). The fluidic electrode channels were filled with phosphate-buffered saline (PBS) solution of conductivity 1.4 S/m and pipette tip reservoirs filled with PBS were inserted into the fluid electrode channel inlet and outlet. Aluminum electrodes connected to the low frequency electronics were inserted into the fluidic electrode reservoirs. After priming, a syringe pump (PHD Ultra, Harvard Apparatus, Holliston, Mass., USA) was used to supply the flow rate of 0.005 mL/hr during the experiments.

To generate the AC electric field, the output signal from a function generator (GFG-3015, GW Instek, Taipei, Taiwan) was amplified (Model AL-50 HF-A/VT, West Nyack, N.Y., USA) to produce output voltages ranging from 0-200VRMS at frequencies between 5 and 70 kHz. Voltage and frequency were monitored using an oscilloscope (TDS-1002B, Tektronics Inc. Beaverton, Oreg., USA) connected to the output of the function generator.

An inverted light microscope (Leica DMI 6000B, Leica Microsystems, Bannockburn, Ill.) equipped with a digital camera (Leica Microsystems) was used for monitoring cells in the main channel, and Leica Application Suite 3.8 software (Leica Microsystems) was used for recording videos of cell response at varied frequencies. Microdevices were kept under vacuum for 30 minutes prior to priming the sample channel with cell suspension and fluid electric channels with PBS. The cell suspension was pumped through the sample channel at 0.005 ml/hr with a syringe pump.

Image processing was accomplished using MATLAB (R2012a, MathWorks Inc., Natick, Mass., USA). For each two minute video, the spatial distribution of cells through the sample channel was determined by recording the position of each cell as it passed a superimposed vertical line. The centerline of each distribution was then compared to the average centerline of control cell distributions (to which no electric field was applied) and the crossover frequency was found by interpolating.

Computational Modeling

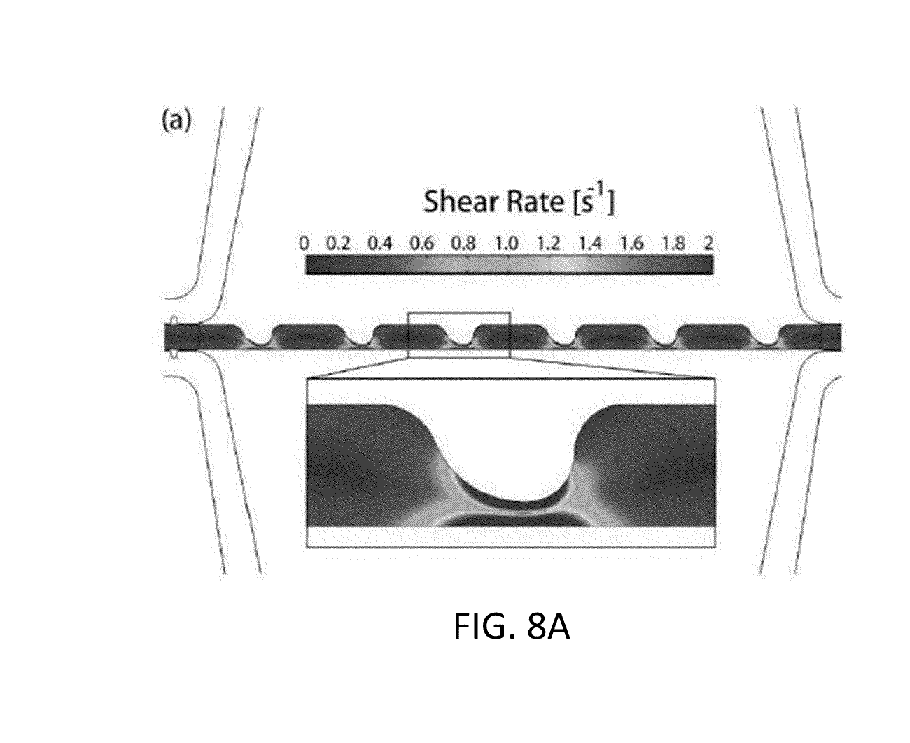

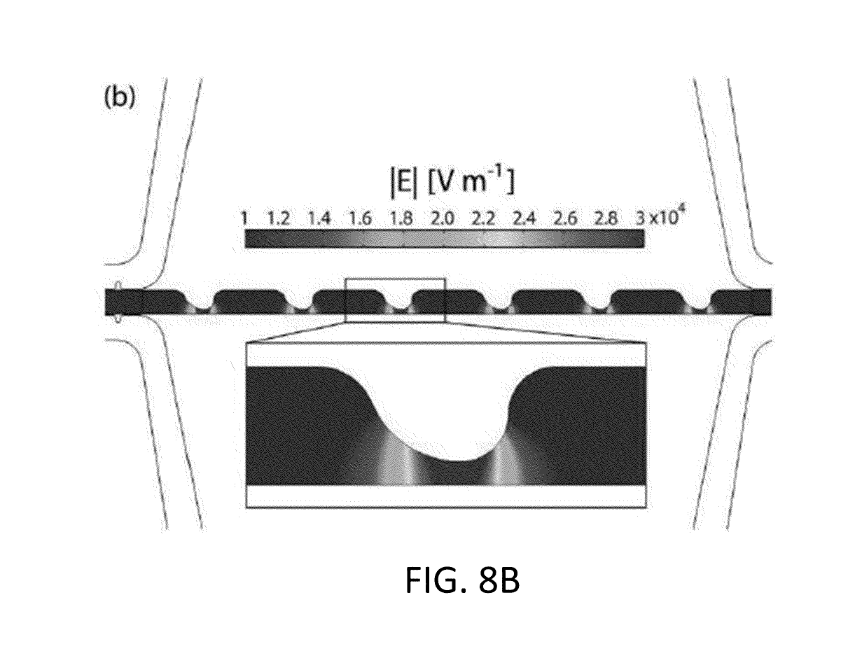

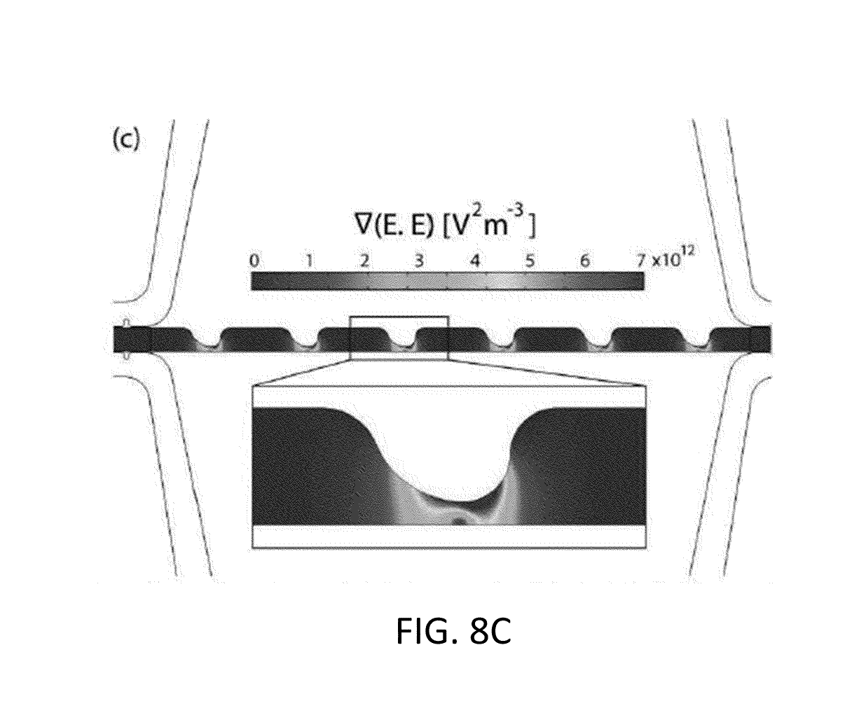

Device performance for the low frequency single-layer device was modeled computationally (FIG. 8A-8C), as was performance for new curved multilayer devices (FIG. 6B, 7B). DEP force (Eqn. 1) was predicted using the Electric Currents module and the shear rate and fluid flow were modeled with the Laminar Flow module of COMSOL Multyphysics 4.3a (Comsol Inc., Burlington, Mass., USA).

\vec{F}_{DEP}=2πα³∈ₘRe[K(ω)]∇(E·E)

Table 1 presents the values of electrical conductivity and permittivity used in the computational modeling. PBS properties were applied to the fluid electrode channels and DEP buffer properties were used for the sample channel. The electrical properties of PDMS used in the model have been reported by the manufacturer (Sylgard 184, Dow Corning, USA). The viscosity and density of water, 0.001 Pa·s and 1000 kg/m³, respectively, were used as the viscosity and density of the sample in the main fluidic channel, given the characteristics of DEP buffer.

TABLE 1
Electrical properties of the materials
used in the computational modeling.

| Material | Electrical conductivity [S/m] | Relative permittivity |
|---|---|---|
| PDMS | 0.83 × 10⁻¹² | 2.65 |
| PBS | 1.4 | 80 |
| DEP buffer | 0.01 | 80 |

FIG. 8A illustrates the shear rate inside the sample channel. The inlet velocity was set to 56 μm/s based on the experimental flow rate of 0.005 mL/hr. The outlet boundary was set to no viscous stress (Dirichlet condition for pressure). No slip boundary conditions were applied to the walls of the sample channel. Then, the Navier-Stokes equations were solved for an incompressible laminar flow. Thus, the maximum shear rate is significantly lower than the shear rate threshold (approximately 5000 s-1) that can cause cell lysis [53, 54]. To model the electric field, uniform potentials and ground at the source and sink fluid electrode channels, respectively, were applied as the boundary conditions. The governing equation ∇·(σ*∇φ)=0, where σ*=σ+iω∈ represents the complex conductivity, was solved to yield the potential distribution, φ. FIGS. 8B and 8C present the magnitude of the electric field and the gradient ∇(\vec{E}_{RMS}·\vec{E}_{RMS}) inside the sample channel, respectively.

Mesh was refined in the sample channel where sawtooth features are located. A mesh resolution study was performed to ensure that the computational results were mesh-independent. To do so, the mesh was refined and compared to the results of previous iteration. Mesh refinement continued until there were maximum 0.01% and 2% differences in the computed values of φ and ∇(\vec{E}_{RMS}·\vec{E}_{RMS}) respectively, compared to the previous iteration.

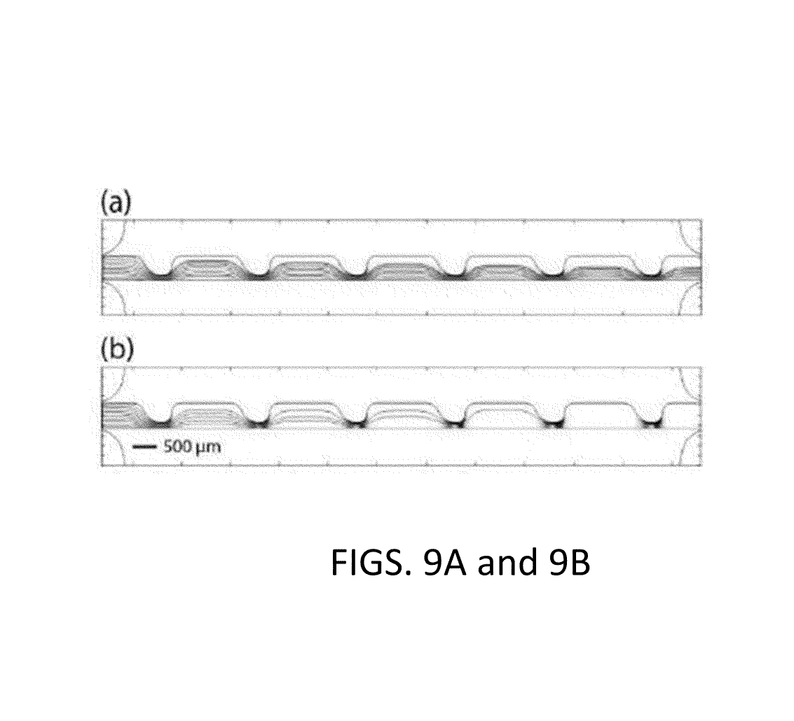

The Particle Tracing for Fluid Flow module was used to predict the trajectories of particles at different frequencies. Trajectories of 10 particles with uniform initial position distribution were simulated (FIGS. 9A and 9B). Drag and DEP forces were added to the model using velocity and electric fields computed from Laminar Flow and Electric Currents modules. The simulations were based on untreated MOSE-L cell properties. Since DEP and drag forces are both proportional to the size of the cells, the smallest cell radius, 5.85 μm, reported previously [29] was used in the simulations. Also, Re[K(ω)] at 5 and 20 kHz were estimated as −0.37 and 0.36, respectively, from the Re[K(ω)] graph reported previously [25]. FIGS. 9A and 9B demonstrate cells trajectories at 5 and 20 kHz, respectively. At frequencies less than the crossover frequency, cells experience nDEP, thus they are repelled from higher ∇(\vec{E}_{RMS}·\vec{E}_{RMS}) and move towards the bottom half of the sample channel (FIG. 9A). At frequencies higher than the crossover frequency cells experience pDEP, thus they are attracted towards higher ∇(\vec{E}_{RMS}·\vec{E}_{RMS}) and the top half of the sample channel (FIG. 9B). In FIG. 9B, some particle trajectories meet the top wall of the sample channel. The plot does not continue to display these trajectories, leading to the appearance of fewer trajectories down the channel.

Results and Discussion

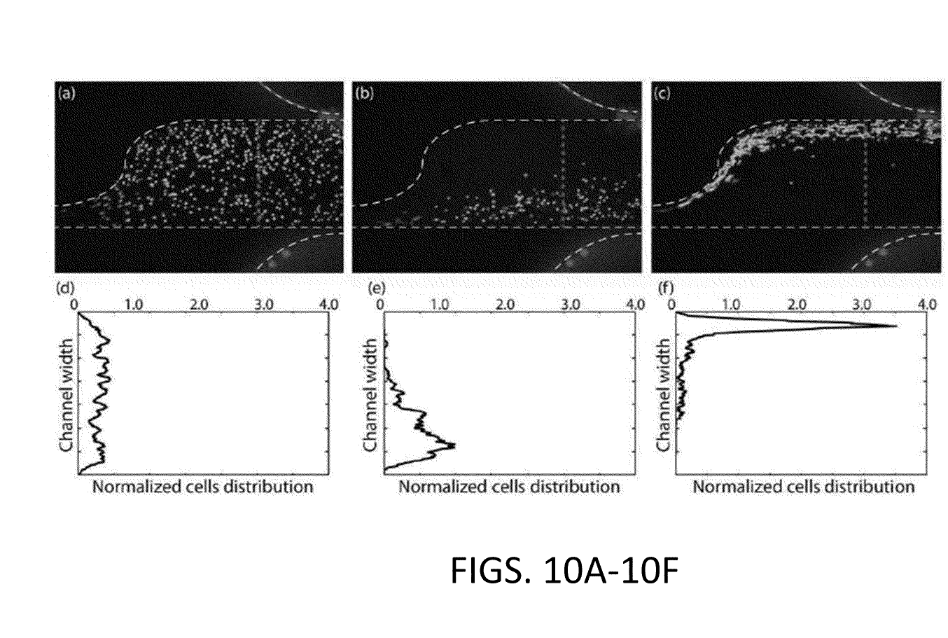

FIGS. 10A, 10B and 10C demonstrate cell movement in the sample channel without any applied electric field, and due to applying 200 VRMS and at frequencies lower and higher than the crossover frequency, respectively. As was shown in the computational results, ∇(\vec{E}_{RMS}·\vec{E}_{RMS}) is much greater at the top side of the channel due to the sawtooth features, which induce nonuniformities into the electric field. When applying a frequency less than the first crossover frequency of cells, cells will experience a negative DEP force and will be repelled from the sawtooth features. Then, they will move towards the bottom half of the channel. However, when applying a frequency higher than the first crossover frequency, cells will experience pDEP force and will be attracted towards sawtooth features and the top side of the channel. FIGS. 10D-F demonstrate the normalized cells distribution corresponding to no DEP force from FIG. 10A, nDEP from FIG. 10B, and pDEP from FIG. 10C, respectively. FIG. 10D shows the distribution of cells without an applied voltage to verify that the cells were randomly distributed in the absence of an electric field. Cell distributions were normalized by the total number of cells crossing the red line in FIGS. 10A-10C to make comparing cells distributions in different experiments possible since the number of cells crossing the line is not exactly equal in all of the experiments. The results presented in FIGS. 10B and C are in agreement with the computational modeling of the trajectories of particles at 5 and 20 kHz.

As shown in FIGS. 10A-10F cells experience a stronger pDEP force than nDEP and they are focused in a narrower stream at the top side of the channel while experiencing pDEP force than when they experience nDEP force, due to two reasons. First, since K(ω) is constrained between −0.5 and 1, the maximum possible value of pDEP force, regardless of the applied frequency, is twice stronger than the nDEP force. Also, ∇(\vec{E}_{RMS}·\vec{E}_{RMS}) increases as the applied frequency is increased, and because, pDEP for cells occurs at higher frequencies than nDEP, cells experience a stronger DEP force during pDEP than nDEP.

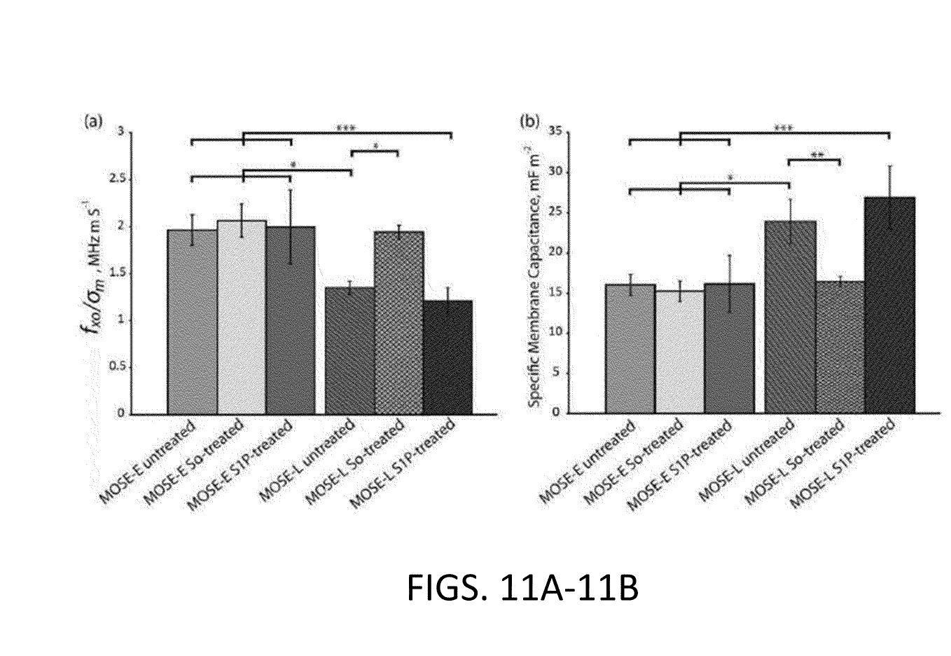

The average crossover frequency for the benign MOSE-E and malignant MOSE-L cells under each treatment condition was calculated. Since the sample conductivity of each cell sample was slightly different, the crossover frequency from each experiment was divided by the sample conductivity in that experiment, based on the linear relationship between conductivity of the sample and crossover frequency (Equation (10)). These values, fₓₒ/σ_{m·}, were compared by a student t-test (FIG. 11A). The ratio of crossover frequencies to sample conductivity, fₓₒ/σ_{m·}, for untreated, So-treated, and S1P-treated MOSE-E cells were 1.96±0.16, 2.06±0.18, and 2.00±0.39 MHz·m/S, respectively, which were not statistically different, indicating that exogenous sphingolipids do not affect the crossover frequencies of MOSE-E. Under identical treatment conditions, fₓₒ/σ_{m·} of MOSE-L cells were 1.35±0.07, 1.94±0.07, and 1.21±0.14 MHz·m/S, respectively. fₓₒ/σ_{m·} for So-treated MOSE-L cells was significantly higher than the control or S1P treated MOSE-L cells (p<0.001). Importantly, there was no statistically significant difference between fₓₒ/σ_{m·} of So-treated MOSE-L cells and control MOSE-E cells (p=0.29), indicating that So treatment effectively reversed the crossover frequency of MOSE-L cells to that observed in MOSE-E cells. The crossover frequency of MOSE-L cells did not change after the treatment with S1P, indicating that the change in electrical properties was due to the So or its metabolites rather than the conversion to S1P or a generic reaction to sphingolipid treatment.

Given the conductivity of the media and the known crossover frequency and radius of the cells, the specific membrane capacitance, C_{mem·}, can be calculated using Equation (10). For MOSE-E control, So or S1P-treated cells, C_{mem·} was 16.05±1.28, 15.26±1.38, and 16.15±3.55 mF/m2, and for MOSE-L cells with identical treatments, C_{mem·} was found to be 23.94±2.75, 16.46±0.62, and 26.89±3.91 mF/m², respectively. Neither So nor S1P treatment caused a significant change in C_{mem·} of MOSE-E cells. The specific membrane capacitance of MOSE-L cells was significantly higher (p<0.01) than MOSE-E cells; treatment with So, however, significantly decreased C_{mem·} to the levels of MOSE-E cells while S1P treatment was not associated with a change in C_{mem·} of MOSE-L cells (FIG. 11B). The results indicate that the decrease in C_{mem·} is specific for So treatment of aggressive cancer cells and benign cells are not affected. the measured radius of 7.185±1.004 and 7.050±1.195 μm of the MOSE-E and MOSE-L cells, respectively, were used to calculate C_{mem·}.

The following discussion explores possible physiological sources for the observed properties, although currently, the underlying events that determine these changes in the dielectric properties during cancer progression are unknown. The specific membrane capacitance of cells can be elevated by an increase in surface protrusions, roughness, and membrane ruffling, traits known to manifest with progressing malignancy, invasiveness, and metastatic potential [55]. This has been shown for leukemia, breast cancer lines, transformed rat kidney, murine erythroleukemia, and oral cancer cells [56, 57, 48, 58]. Consistent with these studies, the inventors observed an elevated specific membrane capacitance with progressing malignancy of MOSE cells (FIG. 11B).

Along these lines, Gascoyne et al [59, 60] defined a membrane-specific area parameter, φ, the ratio of the actual membrane area to the membrane area that would be required to cover a smooth cell with the same radius. Thus, φ can be defined as φ=Cₘₑₘ/C₀, where C₀ is the membrane capacitance of a smooth cell, approximately C₀=9 mF/m^{2.61}. The amount of surface folding and protrusions, and morphological features such as microvilli, villi, ruffles, ridges, and blebs are quantified by φ [59]. These complexities increase the membrane surface area and consequently the membrane capacitance. Cells with irregular surfaces will have φ greater than unity, while a perfectly smooth cell will have φ=1. In the current study, φ increases from 1.78±0.14 for MOSE-E cells to 2.66±0.31 for untreated MOSE-L cells (p<0.001), based on the results presented in FIG. 11B, demonstrating that malignant cells have more surface irregularities than early cells. In their previous study the inventors also showed that φ for MOSE-I cells is 2.01±1.61, which is in between φ values of MOSE-E and MOSE-L cells [62].

After treating MOSE-L cells with So, φ decreased to 1.83±0.07, which is statistically significantly different (p<0.01) from untreated MOSE-L cells. However, treatment of MOSE-L cells with S1P increased φ to 2.99±0.43 (p=0.06) which is an indicator of an increased surface roughness associated with S1P treatment.

To relate the membrane properties of suspended cells to cells in an attached state, Gascoyne recently measured Cₘₑₘ and φ of the cell lines in the NCl-[60] panel, [63] and also examined the exterior morphology of these cell lines by defining a membrane area morphological score, M. M includes three characteristics of cells when are attached in cell culture flask: flatteningon the culture flask surface, cell elongation and the long dendritic projections, and small features, such as ruffles, folds and microvilli on cell surface [63]. They also showed that there is a correlation between φ and M which means that the cells DEP characteristics depend not only on cells size and morphology when suspended, but also on the exterior morphology of cell before releasing from the site of origin or cell culture flask [63]. It was shown previously that MOSE-E cells exhibit a more cobblestone like appearance, whereas the cells take on amore spindle-like morphology as they subsequently progress to more aggressive phenotypes [26]. This observation indicates that M, membrane area morphological score, increases during cancer progression and results, consequently, in increasing φ and changes in dielectric properties of cells, consistent with the inventors' findings.

Changes in dielectric properties of MOSE cells during cancer progression might also result from dysregulation of the cytoskeleton [25]. This dysregulation is common in cancer progression and alters the cellular architecture of cancer cells, affecting cellular functions, growth, and signaling events. The MOSE cell model recapitulates these changes in cellular architecture: MOSE-E cells have well-organized, long, cable-like bundles of actin fibers while MOSE-L cells have a highly disorganized actin and microtubule cytoskeleton [26, 27] critical for the viscoelasticity of the cells [28]. Stage-dependent, step-wise changes in gene expression levels during MOSE neoplastic progression have been reported previously by using mouse whole genome microarray and gene ontology analyses [26, 27]. Specifically, progression was associated with a significant change in the expression or subcellular distribution of key cytoskeletal regulatory proteins, including focal adhesion kinase, α-actinin, and vinculin [27]. Moreover, after treating MOSE-L cells with So, a significant change in the expression levels of these proteins was observed (unpublished observations). These observations are in agreement with the noted changes in dielectric properties of MOSE derived cancer cells and suggest that the dielectric properties of cells could be correlated to a cell gene expression profile [63]. Sphingolipid metabolites have been shown to be involved in the regulation of the cytoskeleton architecture [64, 65] and this phenomenon has recently been confirmed in MOSE derived cancer cells: treatment with So, but not S1P, was associated with an increased organization of the actin stress fibers [27] and increased mechanical stiffness of MOSE-L cells. In contrast, S1P treated cells demonstrated more microvilli-like protrusions on the cell surface (unpublished observations) which may have contributed to the calculated φ increase in MOSE-L cells following S1P treatment (φ=2.66±0.31 for untreated MOSE-L). Overall, the observed shift in dielectric properties of So-treated MOSE-L cells towards a more benign-like MOSE-E profile appears consistent with the inventors' previous findings indicating direct associations between changes in cytoskeleton architecture [26, 27] elasticity [28], and dielectric properties [25, 29] throughout progression, and the effects of sphingolipids on MOSE cell morphology.

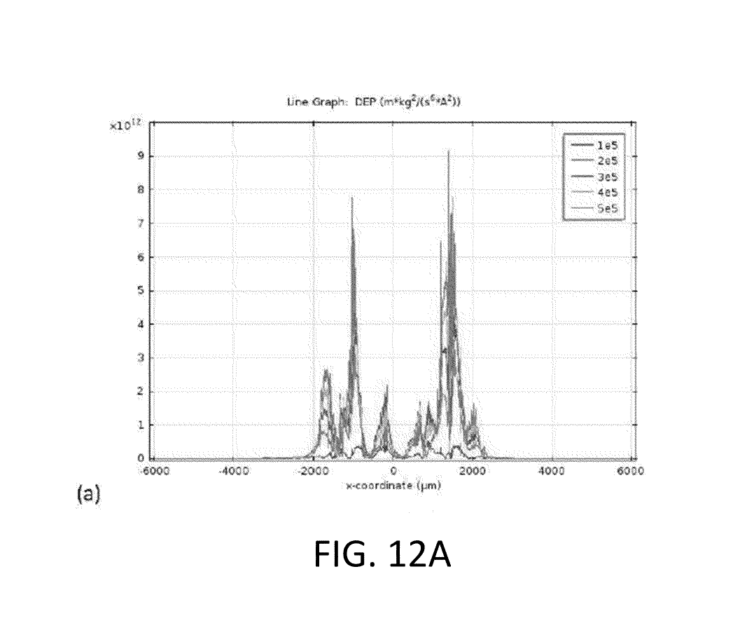

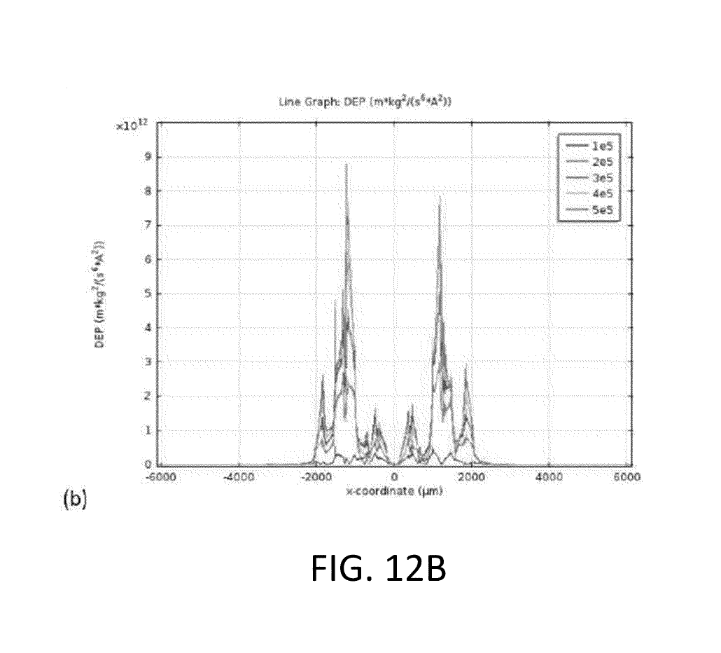

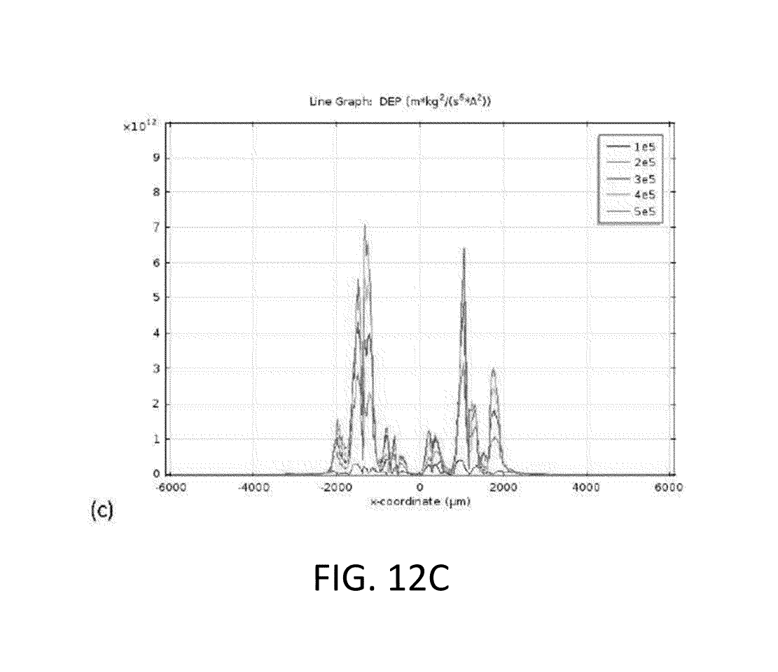

Additionally, it has been found that a tapered electrode channel width results in increasing electric field gradients from bottom to top of the channel (FIG. 12A-12C). By changing the angle of resultant force, and magnitude of the DEP force, it is expected that smaller particles that require greater ∇(E·E) to generate sufficient DEP force to overcome the drag force can be deflected in a stream at the top of the channel while larger particles that require less ∇(E·E) for the DEP force to overcome the drag force will be deflected to a stream nearer the bottom of the channel. Beyond sorting by size, particles with varying biophysical properties should be sorted into streams based on differential DEP forces experienced across the channel. The inventors suggest that this curved design will allow sorting of multiple particles in a “rainbow”-type fashion, where multiple sorted streams can flow to >2 outlets for collection.

FIGS. 12A-12C. Computational modeling of ∇(E·E) at z=35 μM (a) toward top of channel (y=200 μm), (b) at center of channel (y=0 μm), and (c) toward bottom of channel (y=−200 μm). ∇(E·E) increases with increasing y-position in the channel due to narrowing width of the electrode channels.

### CONCLUSIONS

The inventors investigated the effect of non-toxic concentrations of the sphingolipid metabolites, So, a potential anti-cancer agent, and S1P, which is regarded as tumor promoting, on the intrinsic electrical properties of benign and aggressive stages of ovarian cancer. The results show that in contrast to S1P treatment, So treatment correlates with a partial reversal of the aggressive phenotype of late-stage ovarian cancer cells defined by a shift (decrease) in the membrane specific capacitance of MOSE-L cells towards that observed for less aggressive cells. In addition, S1P increased surface membrane protrusions whereas SL-treated cells overall exhibited a smoother surface. The basis of these results is in agreement with previous results showing that the specific membrane capacitance of cells increases during ovarian cancer progression in a synergic model of ovarian cancer cells [25].

These studies suggest that the electrical properties of cancer cells can be targets of cancer preventive and promoting efforts. Future studies need to correlate these changes with the tumorigenicity of the cells and structural and molecular events for the design of effective prevention and treatment strategies. It is possible that cDEP may be used to not only detect cancer cells of different stages but also determine the effectiveness and predict the success of chemopreventive drugs. For instance, the effectiveness of So or conventional chemotherapeutic drugs that impact the cells' surface topography and the actin cytoskeleton may be ascertained by monitoring changes in the cells' electrical signature. The underlying molecular or structural alterations responsible for the changes in dielectric properties and the response to treatment may be critical for the design of devices for cancer detection and treatment control. This would be an advantage over methods that rely solely upon expressed surface receptors, not only for applications such as cell identification and enrichment but also for targeted treatments. Utilizing cDEP for mapping electrical properties of treated cancer cells to specific disease stages of non-treated cells may allow a new, rapid method for determining drug efficacy and for performing dosage studies.

### REFERENCES

1. Visintin I, Feng Z, Longton G, Ward D C, Alvero A B, Lai Y, Tenthorey J, Leiser A, Flores-Saaib R, Yu H, Azori M, Rutherford T, Schwartz P E, Mor G. Clinical cancer research: an official journal of the American Association for Cancer Research. 2008; 14:1065-1072. [PubMed: 18258665]
2. Jacobs I J, Menon U. Molecular & cellular proteomics: MCP. 2004; 3:355-366. [PubMed:14764655]
3. Pohl, H A. Dielectrophoresis. Cambridge University Press; 1978.
4. Shim S, Gascoyne P, Noshari J, Hale K S. Integrative biology: quantitative biosciences from nano to macro. 2011; 3:850-862. [PubMed: 21691666]
5. Vykoukal D M, Gascoyne P R, Vykoukal J. Integrative biology: quantitative biosciences from nano to macro. 2009; 1:477-484. [PubMed: 20023758]
[3A] A. Jemal, R. Siegel, J. Xu, and E. Ward, “Cancer statistics, 2010,” CA Cancer J Clin, vol. 60, pp. 277-300, September-October 2010.
[4A] “StatBite: Ovarian cancer: risk of recurrence by stage of diagnosis,” J Natl Cancer Inst, vol. 101, p. 1234, Sep. 16 2009.
[5A] S. Raguz and E. Yague, “Resistance to chemotherapy: new treatments and novel insights into an old problem,” Br J Cancer, vol. 99, pp. 387-91, Aug. 5, 2008.
6. Gagnon Z R. Electrophoresis. 2011; 32:2466-2487. [PubMed: 21922493]
7. Gagnon Z, Mazur J, Chang H C. Lab Chip. 2010; 10:718-726. [PubMed: 20221559]
8, 14A. Lostumbo A, Mehta D, Setty S, Nunez R., “Flow cytometry: a new approach for the molecular profiling of breast cancer,” Experimental and molecular pathology. 2006; 80:46-53. [PubMed: 16271361]
8A. A. Salmanzadeh, E. S. Elvington, P. C. Roberts, E. M. Schmelz, and R. V. Davalos, “Sphingolipid metabolites modulate dielectric characteristics of cells in a mouse ovarian cancer progression model,” Integr Biol (Camb), Apr. 23 2013.
9, 15A. K. Kato and A. Radbruch, “Isolation and characterization of CD34+ hematopoietic stem cells from human peripheral blood by high-gradient magnetic cell sorting,” Cytometry, 1993; 14:384-392. [PubMed: 7685679]
[9A] H. A. Pohl and J. S. Crane, “Dielectrophoresis of cells,” Biophysical Journal, vol. 11, pp. 711-727, 1971 1971.
10. Hsiung L C, Chiang C L, Wang C H, Huang Y H, Kuo C T, Cheng J Y, Lin C H, Wu V, Chou H Y, Jong D S, Lee H, Wo A M. Lab Chip. 2011; 11:2333-2342. [PubMed: 21629948]
[10A] R. Pethig, “Review Article-Dielectrophoresis: Status of the theory, technology, and applications,” BIOMICROFLUIDICS, vol. 4, June 2010.
11. Labeed F H, Coley H M, Thomas H, Hughes M P. Biophys J. 2003; 85:2028-2034. [PubMed:12944315]
12. Coley H M, Labeed F H, Thomas H, Hughes M P. Bba-Gen Subjects. 2007; 1770:601-608.
[12A] Z. Gagnon, J. Mazur, and H. C. Chang, “Glutaraldehyde enhanced dielectrophoretic yeast cell separation,” Biomicrofluidics, vol. 3, p. 44108, 2009.
13. Hubner Y, Hoettges K F, Kass G E N, Ogin S L, Hughes M P. lee P-Nanobiotechnol. 2005; 152:150-154.
[13A] I. F. Cheng, H. C. Chang, and D. Hou, “An integrated dielectrophoretic chip for continuous bioparticle filtering, focusing, sorting, trapping, and detecting,” Biomicrofluidics, vol. 1, p. 21503, 2007.
14. Khoshmanesh K, Akagi J, Nahavandi S, Skommer J, Baratchi S, Cooper J M, Kalantar-Zadeh K, Williams D E, Wlodkowic D. Anal Chem. 2011; 83:2133-2144. [PubMed: 21344868]
15. Pethig R, Bressler V, Carswell-Crumpton C, Chen Y, Foster-Haje L, Garcia-Ojeda M E, Lee R S, Lock G M, Talary M S, Tate K M. Electrophoresis. 2002; 23:2057-2063. [PubMed: 12210259]
16, 43A. H. Shafiee, J. Caldwell, M. Sano, and R. Davalos, “Contactless dielectrophoresis: a new technique for cell manipulation,” Biomedical Microdevices, vol. 11, pp. 997-1006, 2009. [PubMed: 19415498]
17, 20A. H. Shafiee, M. B. Sano, E. A. Henslee, J. L. Caldwell, and R. V. Davalos, “Selective isolation of live/dead cells using contactless dielectrophoresis (cDEP),” Lab on a Chip, vol. 10, pp. 438-445, 2010. [PubMed: 20126683]
18. Salmanzadeh A, Shafiee H, Davalos R V, Stremler M A. Electrophoresis. 2011; 32:2569-2578.[PubMed: 21922498]
[18A] Y. Huang, S. Joo, M. Duhon, M. Heller, B. Wallace, and X. Xu, “Dielectrophoretic cell separation and gene expression profiling on microelectronic chip arrays,” Analytical Chemistry, vol. 74, pp. 3362-3371, Jul. 15 2002.
19. Sano M B, Henslee E A, Schmelz E, Davalos R V. Electrophoresis. 2011; 32:3164-3171. [PubMed:22102497]
[19A] M. Stephens, M. S. Talary, R. Pethig, A. K. Burnett, and K. I. Mills, “The dielectrophoresis enrichment of CD34(+) cells from peripheral blood stem cell harvests,” Bone Marrow Transplantation, vol. 18, pp. 777-782, October 1996.
20. Sano M B, Salmanzadeh A, Davalos R V. Electrophoresis. 2012; 33:1938-1946. [PubMed:22806458]
21. Salmanzadeh A, Romero L, Shafiee H, Gallo-Villanueva R C, Stremler M A, Cramer S D, Davalos R V. Lab Chip. 2011; 12:182-189. [PubMed: 22068834]
[21A] R. V. Davalos, H. Shafiee, M. B. Sano, and J. L. Caldwell, “Devices and Methods for Contactless Dielectrophoresis For Cell or Particle Manipulation,” United States Patent Publication US 2010/0224493.
22. Sano M B, Caldwell J L, Davalos R V. Biosensors and Bioelectronics. 2011; 30:13-20. [PubMed:21944186]
23. Salmanzadeh, A.; Sano, M B.; Shafiee, H.; Stremler, M A.; Davalos, R V. presented in part at the EMBC; San Diego, Calif. 2012;
24. Henslee E A, Sano M B, Rojas A D, Schmelz E M, Davalos R V. Electrophoresis. 2011; 32:2523-2529. [PubMed: 21922494]
[24A] A. N. Ketene, E. M. Schmelz, P. C. Roberts, and M. Agah, “The effects of cancer progression on the viscoelasticity of ovarian cell cytoskeleton structures,” Nanomedicine, Jun. 23, 2011.
25, 6A. A. Salmanzadeh, M. B. Sano, R. C. Gallo-Villanueva, P. C. Roberts, E. M. Schmelz, and R. V. Davalos, “Investigating dielectric properties of different stages of syngeneic murine ovarian cancer cells,” Biomicrofluidics, vol. 7, January 2013 [011809].
26, 22A. P. C. Roberts, E. P. Mottillo, A. C. Baxa, H. H. Heng, N. Doyon-Reale, L. Gregoire, W. D. Lancaster, R. Rabah, and E. M. Schmelz, “Sequential molecular and cellular events during neoplastic progression: a mouse syngeneic ovarian cancer model,” Neoplasia, vol. 7, pp. 944-56, October 2005. [PubMed: 16242077]
[26A] H.-S. Moon, K. Kwon, S.-I. Kim, H. Han, J. Sohn, S. Lee, and H.-I. Jung, “Continuous separation of breast cancer cells from blood samples using multi-orifice flow fractionation (MOFF) and dielectrophoresis (DEP)” Lab on a Chip, vol. 11, pp. 1118-1125, 2011.
27, 23A. A. L. Creekmore, W. T. Silkworth, D. Cimini, R. V. Jensen, P. C. Roberts, and E. M. Schmelz, “Changes in gene expression and cellular architecture in an ovarian cancer progression model,” PLoS ONE, vol. 6, p. e17676, 2011.
[27A] I.-F. Cheng, V. E. Froude, Y. Zhu, H.-C. Chang, and H.-C. Chang, “A continuous high-throughput bioparticle sorter based on 3D traveling-wave dielectrophoresis,” Lab on a Chip, vol. 9, pp. 3193-3201, 2009.
28. Ketene A N, Roberts P C, Shea A A, Schmelz E M, Agah M. Integrative Biology. 2012; 4:540-549. [PubMed: 22446682]
[28A] S. Agastin, M. R. King, and T. B. Jones, “Rapid enrichment of biomolecules using simultaneous liquid and particulate dielectrophoresis,” Lab on a Chip, vol. 9, pp. 2319-2325, 2009.
29, 7A. A. Salmanzadeh, H. Kittur, M. B. Sano, C. R. P, E. M. Schmelz, and R. V. Davalos, “Dielectrophoretic differentiation of mouse ovarian surface epithelial cells, macrophages, and fibroblasts using contactless dielectrophoresis,” Biomicrofluidics, vol. 6, pp. 24104-24113, June 2012. [PubMed: 22536308]
[29A] J. Vykoukal, D. M. Vykoukal, S. Freyberg, E. U. Alta, and P. R. C. Gascoyne, “Enrichment of putative stem cells from adipose tissue using dielectrophoretic field-flow fractionation,” Lab on a Chip, vol. 8, pp. 1386-1393, 2008.
30. Schmelz E M, Roberts P C, Kustin E M, Lemonnier L A, Sullards M C, Dillehay D L, Merrill A H Jr. Cancer Res. 2001; 61:6723-6729. [PubMed: 11559543]
[30A] J. Cheng, E. Sheldon, L. Wu, A. Uribe, L. Gerrue, J. Carrino, M. Heller, and J. O'Connell, “Preparation and hybridization analysis of DNA/RNA from E. coli on microfabricated bioelectronic chips,” Nat Biotechnol, vol. 16, pp. 541-546, 1998.
31. Schmelz E M, Sullards M C, Dillehay D L, Merrill A H Jr. J. Nutr. 2000; 130:522-527. [PubMed:10702579]
[31A] P. Gascoyne, C. Mahidol, M. Ruchirawat, Jutamaad, Satayavivad, P. Watcharasit, and F. F. Becker, “Microsample preparation by dielectrophoresis: isolation of malaria,” Lab on a Chip, vol. 2, pp. 70-75, 2002.
32. Lemonnier L A, Dillehay D L, Vespremi M J, Abrams J, Brody E, Schmelz E M. Arch Biochem Biophys. 2003; 419:129-138. [PubMed: 14592456]
33. Simon K W, Tait L, Miller F, Cao C, Davy K P, Leroith T, Schmelz E M. Food Funct. 2010; 1:90-98. [PubMed: 21776459]
[33A] T. B. Jones, “Basic theory of dielectrophoresis and electrorotation,” leee Engineering in Medicine and Biology Magazine, vol. 22, pp. 33-42, 2003 2003.
34. Mazzei J C, Zhou H, Brayfield B P, Hontecillas R, Bassaganya-Riera J, Schmelz E M. J Nutr Biochem. 2011
[34A] H. A. Pohl and K. Pollock, “Dielectrophoretic Cell Sorting,” Journal of the Electrochemical Society, vol. 130, pp. C121-C121, 1983.
35. Gault C R, Obeid L M, Hannun Y A. Adv Exp Med. Biol. 2010; 688:1-23. [PubMed: 20919643]
36. Gangoiti P, Camacho L, Arana L, Ouro A, Granado M H, Brizuela L, Casas J, Fabrias G, Abad J L, Delgado A, Gomez-Munoz A. Prog Lipid Res. 2010; 49:316-334. [PubMed: 20193711]
37. Kolesnick R, Golde D W. Cell. 1994; 77:325-328. [PubMed: 8181053]
[37A] A. Salmanzadeh, L. Romero, H. Shafiee, R. C. Gallo-Villanueva, M. A. Stremler, S. D. Cramer, and R. V. Davalos, “Isolation of prostate tumor initiating cells (TICs) through their dielectrophoretic signature,” Lab on a Chip, v. 12, p. 182-9, Jan. 7, 2012.
38. Kolesnick R N, Hemer M R. J Biol. Chem. 1990; 265:18803-18808. [PubMed: 2172234]
[38A] M. J. Madou, Fundamentals of Microfabrication and Nanotechnology, Third Edition, Three-Volume Set. Irvine, USA CRC Press, 1992.
39. Spiegel S, Milstien S, Nat Rev Mol Cell Bio. 2003; 4:397-407. [PubMed: 12728273]
40. Maceyka M, Payne S G, Milstien S, Spiegel S. Bba-Mol Cell Biol L. 2002; 1585:193-201.
[40A] B. Eker, R. Meissner, A. Bertsch, K. Mehta, and P. Renaud, “Label-Free Recognition of Drug Resistance via Impedimetric Screening of Breast Cancer Cells,” PLoS ONE, vol. 8, 2013 Mar. 4, 2013.
41. Chalfant, C.; Del Poeta, M. Sphingolipids as signaling and regulatory molecules. Springer Science +Business Media; Landes Bioscience; New York, N.Y. Austin, Tex.: 2010.
[41A] Y. Hu and G. K. Smyth, “ELDA: extreme limiting dilution analysis for comparing depleted and enriched populations in stem cell and other assays,” Journal of immunological methods, vol. 347, pp. 70-78, 2009 Aug. 15, 2009.
42. Teichgraber V, Ulrich M, Endlich N, Riethmuller J, Wilker B, De Oliveira-Munding C C, van Heeckeren A M, Barr M L, von Kurthy G, Schmid K W, Weller M, Tummler B, Lang F, Grassme H, Doring G, Gulbins E. Nature medicine. 2008; 14:382-391.
[42A] C. Cohen, A. Shea, C. Heffron, E. Schmelz, and P. Roberts, “Intra-abdominal fat depots represent distinct immunomodulatory microenvironments: a murine model,” PLoS One, vol. 8, p. e66477, 2013.
43. Wang D, Zhao Z, Caperell-Grant A, Yang G, Mok S C, Liu J, Bigsby R M, Xu Y. Mol Cancer Ther. 2008; 7:1993-2002. [PubMed: 18645009]
44. Visentin B, Vekich J A, Sibbald B J, Cavalli A L, Moreno K M, Matteo R G, Garland W A, Lu Y L, Yu S X, Hall H S, Kundra V, Mills G B, Sabbadini R A. Cancer Cell. 2006; 9:225-238. [PubMed:16530706]
[44A] R. V. Davalos, G. J. McGraw, T. I. Wallow, A. M. Morales, K. L. Krafcik, Y. Fintschenko, E. B. Cummings, and B. A. Simmons, “Performance impact of dynamic surface coatings on polymeric insulator-based dielectrophoretic particle separators,” Analytical and Bioanalytical Chemistry, vol. 390, pp. 847-855, 2008.
45. Irimajiri A, Hanai T, Inouye A. Journal of theoretical biology. 1979; 78:251-269. [PubMed:573830]
[45A] B. A. Simmons, G. J. McGraw, R. V. Davalos, G. J. Fiechtner, Y. Fintschenko, and E. B. Cummings, “The Development of Polymeric Devices as Dielectrophoretic Separators and Concentrators,” MRS Bulletin, v. 31, p. 120-124, 2006.
46. Pethig R, Talary M S. let Nanobiotechnol. 2007; 1:2-9. [PubMed: 17500582]
[46A] M. Urdaneta and E. Smela, “Multiple frequency dielectrophoresis,” Electrophoresis, vol. 28, pp. 3145-3155, 2007 2007.
47. Pethig R. Biomicrofluidics. 2010; 4:022811. [PubMed: 20697589]
48. Huang Y, Wang X B, Becker F F, Gascoyne P R C. Bba-Gen Subjects. 1996; 1282:76-84.
49. Arnold W M, Zimmermann U. J. Electrostat. 1988; 21:151-191.
50. Wang X, Becker F F, Gascoyne P R. Chaos. 2010; 20:043133. [PubMed: 21198103]
51. Wang X B, Huang Y, Gascoyne P R, Becker F F, Holzel R, Pethig R. Biochim Biophys Acta. 1994; 1193:330-344. [PubMed: 8054355]
52, 39A. L. A. Flanagan, J. Lu, L. Wang, S. A. Marchenko, N. L. Jeon, A. P. Lee, and E. S. Monuki, “Unique Dielectric Properties Distinguish Stem Cells and Their Differentiated Progeny,” STEM CELLS, vol. 26, pp. 656-665, 2008 [PubMed: 18096719]
53. Skalak, R C S. Handbook of Bioengineering. McGraw-Hill; NY: 1987.
54. Fung, Y C P N.; Anliker, M. Biomechanics, Its Foundations and Objectives. Prentice Hall; Englewood Cliffs, N.J.: 1972.
55. Jiang W G. European journal of surgical oncology: the journal of the European Society of Surgical Oncology and the British Association of Surgical Oncology. 1995; 21:307-309.
56, 16A. F. Becker, X. Wang, Y. Huang, R. Pethig, J. Vykoukal, and P. Gascoyne, “The removal of human leukemia cells from blood using interdigitated microeletrodes,” Journal of Physics D: Applied Physics, vol. 27, pp. 2659-2662, 1994.
57. Becker F F, Wang X B, Huang Y, Pethig R, Vykoukal J, Gascoyne P R C. P Natl Acad Sci USA. 1995; 92:860-864.
58. Mulhall H J, Labeed F H, Kazmi B, Costea D E, Hughes M P, Lewis M P. Analytical and bioanalytical chemistry. 2011; 401:2455-2463. [PubMed: 21877186]
59, 17A. P. R. C. Gascoyne, X. B. Wang, Y. Huang, and F. F. Becker, “Dielectrophoretic separation of cancer cells from blood,” IEEE Trans Ind Appl, vol. 33, pp. 670-678, May-June 1997. [PubMed: 20011619]
60. Gascoyne P R, Shim S, Noshari J, Becker F F, Stemke-Hale K. Electrophoresis. 2012
61, 32A. W. M. Arnold and U. Zimmermann, “Rotation of an isolated cell in a rotating electric field,” Die Naturwissenschaften, vol. 69, pp. 297-298, 1982/06//1982. [PubMed: 7110365]
62. Salmanzadeh A, Sano M B, Gallo-Villanueva R C, Roberts P C, Schmelz E M, Davalos R V. Biomicrofluidics. in press.
63. Gascoyne P R, Shim S, Noshari J, Becker F F, Stemke-Hale K. Electrophoresis. 2013
64. Strub G M, Maceyka M, Hait N C, Milstien S, Spiegel S. Adv Exp Med Biol. 2010; 688:141-155. [PubMed: 20919652]
65. Hanna A N, Berthiaume L G, Kikuchi Y, Begg D, Bourgoin S, Brindley D N. Mol Biol Cell. 2001; 12:3618-3630. [PubMed: 11694593]

Additional references include: Creekmore, A. L. et al, “Regulation of cytoskeleton organization by sphingosine in a mouse cell model of progressive ovarian cancer,” Biomolecules 3(3): 386-407, 2013; and Babahosseini, H., et al., “Roles of bioactive sphingolipid metabolites in ovarian cancer cell biomechanics,” Conf Proc IEEE Eng Med Biol Soc. 2012; 2012:2436-9.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. An in-vitro method of detecting drug-resistant or responsive cells comprising:
subjecting a suspension of patient cells to an AC electric field in vitro;
measuring a dielectrophoretic response of the patient cells;
comparing the measured dielectrophoretic response to a previous dielectrophoretic response of the patient cells and determining any difference;
using the difference in dielectrophoretic responses to determine if there is an increased drug resistance or responsiveness of the patient cells to a drug.

2. The method of claim 1 comprising:
providing cells from a patient;
preparing a suspension of the patient cells in a fluid medium;
subjecting the suspension of patient cells to an AC electric field in vitro;
measuring a dielectrophoretic response of the patient cells;
treating cells of the patient in vitro with a drug;
providing a second suspension of the drug treated cells;
subjecting the second suspension of cells to an AC electric field in vitro;
measuring a dielectrophoretic response of the drug treated cells;
comparing the dielectrophoretic response of the drug treated cells to the dielectrophoretic response of the patient cells and determining any difference;
using the difference to determine whether the patient cells are responsive to the drug and/or whether the patient cells are resistant to the drug.

3. The method of claim 2, wherein the drug is chosen from any one or more of Abiraterone Acetate, ABITREXATE (Methotrexate), ABRAXANE (Paclitaxel Albumin-stabilized Nanoparticle Formulation), ADCETRIS (Brentuximab Vedotin), Ado-Trastuzumab Emtansine, ADRIAMYCIN (Doxorubicin Hydrochloride), ADRUCIL (Fluorouracil), Afatinib Dimaleate, AFINITOR (Everolimus), ALDARA (Imiquimod), Aldesleukin, Alemtuzumab, ALIMTA (Pemetrexed Disodium), ALOXI (Palonosetron Hydrochloride), AMBOCHLORIN (Chlorambucil), AMBOCLORIN (Chlorambucil), Aminolevulinic Acid, Anastrozole, Aprepitant, AREDIA (Pamidronate Disodium), ARIMIDEX (Anastrozole), AROMASIN (Exemestane), ARRANON (Nelarabine), Arsenic Trioxide, ARZERRA (Ofatumumab), Asparaginase Erwinia chrysanthemi, AVASTIN (Bevacizumab), Axitinib, Azacitidine, Bendamustine Hydrochloride, Bevacizumab, Bexarotene, BEXXAR (Tositumomab and I 131 Iodine Tositumomab), Bleomycin, Bortezomib, BOSULIF (Bosutinib), Cabazitaxel, Cabozantinib-S-Malate, CAMPATH (Alemtuzumab), CAMPTOSAR (Irinotecan Hydrochloride), Capecitabine, Carboplatin, Carfilzomib, CEENU (Lomustine), CERUBIDINE (Daunorubicin Hydrochloride), Cetuximab, Chlorambucil, Cisplatin, CLAFEN (Cyclophosphamide), Clofarabine, COMETRIQ (Cabozantinib-S-Malate), COSMEGEN (Dactinomycin), Crizotinib, Cyclophosphamide, CYFOS (Ifosfamide), Cytarabine, Dabrafenib, Dacarbazine, DACOGEN (Decitabine), Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, EFUDEX (Fluorouracil), ELITEK (Rasburicase), ELLENCE (Epirubicin Hydrochloride), ELOXATIN (Oxaliplatin), Eltrombopag Olamine, EMEND (Aprepitant), Enzalutamide, Epirubicin Hydrochloride, ERBITUX (Cetuximab), Eribulin Mesylate, ERIVEDGE (Vismodegib), Erlotinib Hydrochloride, ERWINAZE (Asparaginase Erwinia chrysanthemi), Etoposide, Everolimus, EVISTA (Raloxifene Hydrochloride), Exemestane, FARESTON (Toremifene), FASLODEX (Fulvestrant), FEMARA (Letrozole), Filgrastim, FLUDARA (Fludarabine Phosphate), Fludarabine Phosphate, FLUOROPLEX (Fluorouracil), Fluorouracil, Folinic acid, FOLOTYN (Pralatrexate), Fulvestrant, Gefitinib, Gemcitabine Hydrochloride, Gemtuzumab Ozogamicin, GEMZAR (Gemcitabine Hydrochloride), GILOTRIF (Afatinib Dimaleate), GLEEVEC (Imatinib Mesylate), HALAVEN (Eribulin Mesylate), HERCEPTIN (Trastuzumab), HYCAMTIN (Topotecan Hydrochloride), Ibritumomab Tiuxetan, ICLUSIG (Ponatinib Hydrochloride), Ifosfamide, Imatinib Mesylate, Imiquimod, INLYTA (Axitinib), INTRON A (Recombinant Interferon Alfa-2b), Iodine 131 Tositumomab and Tositumomab, Ipilimumab, IRESSA (Gefitinib), Irinotecan Hydrochloride, ISTODAX (Romidepsin), Ixabepilone, JAKAFI (Ruxolitinib Phosphate), JEVTANA (Cabazitaxel), Kadcyla (Ado-Trastuzumab Emtansine), KEOXIFENE (Raloxifene Hydrochloride), KEPIVANCE (Palifermin), KYPROLIS (Carfilzomib), Lapatinib Ditosylate, Lenalidomide, Letrozole, Leucovorin Calcium, Leuprolide Acetate, Lomustine, LUPRON (Leuprolide Acetate, MARQIBO (Vincristine Sulfate Liposome), MATULANE (Procarbazine Hydrochloride), Mechlorethamine Hydrochloride, MEGACE (Megestrol Acetate), Megestrol Acetate, MEKINIST (Trametinib), Mercaptopurine, Mesna, METHAZOLASTONE (Temozolomide), Methotrexate, Mitomycin, MOZOBIL (Plerixafor), MUSTARGEN (Mechlorethamine Hydrochloride), MUTAMYCIN (Mitomycin C), MYLOSAR (Azacitidine), MYLOTARG (Gemtuzumab Ozogamicin), Nanoparticle Paclitaxel (Paclitaxel Albumin-stabilized Nanoparticle Formulation), NAVELBINE (Vinorelbine Tartrate), Nelarabine, NEOSAR (Cyclophosphamide), NEUPOGEN (Filgrastim), NEXAVAR (Sorafenib Tosylate), Nilotinib, NOLVADEX (Tamoxifen Citrate), NPLATE (Romiplostim), Ofatumumab, Omacetaxine Mepesuccinate, ONCASPAR (Pegaspargase), ONTAK (Denileukin Diftitox), Oxaliplatin, Paclitaxel, Paclitaxel Albumin-stabilized Nanoparticle Formulation, Palifermin, Palonosetron Hydrochloride, Pamidronate Disodium, Panitumumab, Pazopanib Hydrochloride, Pegaspargase, Peginterferon Alfa-2b, PEG-INTRON (Peginterferon Alfa-2b), Pemetrexed Disodium, Pertuzumab, PLATINOL (Cisplatin), PLATINOL-AQ (Cisplatin), Plerixafor, Pomalidomide, POMALYST (Pomalidomide), Ponatinib Hydrochloride, Pralatrexate, Prednisone, Procarbazine Hydrochloride, PROLEUKIN (Aldesleukin), PROLIA (Denosumab), PROMACTA (Eltrombopag Olamine), PROVENGE (Sipuleucel-T), PURINETHOL (Mercaptopurine), Radium 223 Dichloride, Raloxifene Hydrochloride, Rasburicas, Recombinant Interferon Alfa-2b, Regorafenib, REVLIMID (Lenalidomide), RHEUMATREX (Methotrexate), Rituximab, Romidepsin, Romiplostim, RUBIDOMYCIN (Daunorubicin Hydrochloride), Ruxolitinib Phosphat, Sipuleucel-T, Sorafenib Tosylate, SPRYCEL (Dasatinib), STIVARGA (Regorafenib), Sunitinib Malate, SUTENT (Sunitinib Malate), SYLATRON (Peginterferon Alfa-2b), SYNOVIR (Thalidomide), SYNRIBO (Omacetaxine Mepesuccinate), TAFINLAR (Dabrafenib), Tamoxifen Citrate, TARABINE PFS (Cytarabine), TARCEVA (Erlotinib Hydrochloride), TARGRETIN (Bexarotene), TASIGNA (Nilotinib), TAXOL (Paclitaxel), TAXOTERE (Docetaxel), TEMODAR (Temozolomide), Temozolomide, Temsirolimus, Thalidomide, TOPOSAR (Etoposide), Topotecan Hydrochloride, Toremifene, TORISEL (Temsirolimus), Tositumomab and I 131 Iodine Tositumomab, TOTECT (Dexrazoxane Hydrochloride), Trametinib, Trastuzumab, TREANDA (Bendamustine Hydrochloride), TRISENOX (Arsenic Trioxide), TYKERB (Lapatinib Ditosylate), Vandetanib, VECTIBIX (Panitumumab), VeIP, VELBAN (Vinblastine Sulfate), VELCADE (Bortezomib), VELSAR (Vinblastine Sulfate), Vemurafenib, VEPESID (Etoposide), VIADUR (Leuprolide Acetate), VIDAZA (Azacitidine), Vinblastine Sulfate, Vincristine Sulfate, Vinorelbine Tartrate, Vismodegib, VORAXAZE (Glucarpidase), Vorinostat, VOTRIENT (Pazopanib Hydrochloride), WELLCOVORIN (Leucovorin Calcium), XALKORI (Crizotinib), XELODA (Capecitabine), XGEVA (Denosumab), XOFIGO (Radium 223 Dichloride), XTANDI (Enzalutamide), YERVOY (Ipilimumab), ZALTRAP (Ziv-Aflibercept), ZELBORAF (Vemurafenib), ZEVALIN (Ibritumomab Tiuxetan), ZINECARD (Dexrazoxane Hydrochloride), Ziv-Aflibercept, Zoledronic Acid, ZOLINZA (Vorinostat), ZOMETA (Zoledronic Acid), and ZYTIGA (Abiraterone Acetate).

4. The in-vitro method of claim 3, wherein the AC electric field is generated using one or more of dielectrophoresis (DEP), contactless dielectrophoresis (cDEP) or insulating dielectrophoresis (iDEP).

5. The method of claim 2, further comprising directing patient treatment decisions or treatment modifications based on the difference in dielectrophoretic responses.

6. The method of claim 2, further comprising evaluating a drug for efficacy with respect to a particular patient, where the patient has not been treated with the drug previously.

7. The method of claim 6, wherein efficacy of the drug is exhibited by a difference in dielectrophoretic response that shifts the cells towards a more benign phenotype and wherein inefficacy of the drug is exhibited by a difference in dielectrophoretic response that shifts the cells towards a more aggressive phenotype.

8. A method of providing an enriched suspension of cells comprising:
providing a suspension of cells from heterogeneous tumors;
subjecting the suspension of cells to an AC electric field in vitro;
separating the suspension of cells into sub-populations of cells;
isolating a target sub-population of cells;
optionally analyzing the isolated sub-population of cells by off-chip studies.

9. The in-vitro method of claim 8, wherein the AC electric field is generated using one or more of dielectrophoresis (DEP), contactless dielectrophoresis (cDEP) or insulating dielectrophoresis (iDEP).

10. The in-vitro method of claim 8, wherein the studies are chosen from one or more of genomic/mutational analyses, molecular and biochemical analyses for biomarker development or for drug screening, or analyses for directing ongoing and further treatment of a patient.

11. The in-vitro method of claim 8, wherein the heterogeneous tumors comprise mixed biopsy or bodily fluid populations.

12. The in-vitro method of claim 8, wherein different tumor stages are detected and/or isolated for early detection and/or further analyses.

13. The method of claim 4, wherein cDEP is performed using frequencies below 100 kHz to determine an effect of non-toxic doses of a drug on cells.

14. The method of claim 13, wherein the cells are cancer cells.

15. The method of claim 8, wherein cDEP is performed using frequencies of 100 kHz to 500 MHz.

16. The method of claim 15, wherein cDEP is performed to selectively isolate target cells from a mixture.

17. A method of determining a property of a plurality of cancer cells, comprising:
providing a dielectrophoresis device comprising:
a sample channel for receiving a sample having a separating portion;
a first electrode channel for receiving a first electrode;
a second electrode channel for receiving a second electrode;
a first insulation barrier between the first and second electrode channels and a second insulation barrier between the second electrode channel and the sample channel;
providing a plurality of cancer cells in a cancer cell suspension;
introducing the cancer cell suspension to the sample channel;
generating an AC electric field through the first and second electrode; and
measuring the spatial distribution of cells through the sample channel;
wherein the spatial distribution is characteristic of a property of the cancer cells.

18. The method of claim 17, wherein the property of the cancer cells is sensitivity or resistance to a cancer therapeutic agent.

19. The method of claim 18, wherein the cancer therapeutic agent is selected from one or more of the group consisting of abiraterone acetate, methotrexate, paclitaxel albumin-stabilized nanoparticle formulation, brentuximab vedotin, ado-trastuzumab emtansine, doxorubicin hydrochloride, fluorouracil, afatinib dimaleate, everolimus, imiquimod, aldesleukin, alemtuzumab, pemetrexed disodium, palonosetron hydrochloride, chlorambucil, aminolevulinic acid, anastrozole, aprepitant, pamidronate disodium, anastrozole, exemestane, nelarabine, arsenic trioxide, ofatumumab, asparaginase erwinia chrysanthemi, bevacizumab, axitinib, azacitidine, bendamustine hydrochloride, bevacizumab, bexarotene, tositumomab and i 131 iodine tositumomab, bleomycin, bortezomib, bosutinib, cabazitaxel, cabozantinib-s-malate, alemtuzumab, irinotecan hydrochloride, capecitabine, carboplatin, carfilzomib, lomustine, daunorubicin hydrochloride, cetuximab, chlorambucil, cisplatin, cyclophosphamide, clofarabine, cabozantinib-s-malate, dactinomycin, crizotinib, ifosfamide, cytarabine, dabrafenib, dacarbazine, decitabine, dactinomycin, dasatinib, daunorubicin hydrochloride, decitabine, degarelix, denileukin diftitox, denosumab, dexrazoxane hydrochloride, docetaxel, doxorubicin hydrochloride, fluorouracil, rasburicase, epirubicin hydrochloride, oxaliplatin, eltrombopag olamine, aprepitant, enzalutamide, epirubicin hydrochloride, cetuximab, eribulin mesylate, vismodegib, erlotinib hydrochloride, etoposide, everolimus, raloxifene hydrochloride, exemestane, toremifene, fulvestrant, letrozole, filgrastim, fludarabine phosphate, fluorouracil, folinic acid, pralatrexate, fulvestrant, gefitinib, gemcitabine hydrochloride, gemtuzumab ozogamicin, gemcitabine hydrochloride, afatinib dimaleate, imatinib mesylate, eribulin mesylate, trastuzumab, topotecan hydrochloride, ibritumomab tiuxetan, ponatinib hydrochloride, ifosfamide, imatinib mesylate, imiquimod, axitinib, recombinant interferon alfa-2b, iodine 131 tositumomab and tositumomab, ipilimumab, gefitinib, irinotecan hydrochloride, romidepsin, ixabepilone, ruxolitinib phosphate, cabazitaxel, ado-trastuzumab emtansine, raloxifene hydrochloride, palifermin, carfilzomib, lapatinib ditosylate, lenalidomide, letrozole, leucovorin calcium, leuprolide acetate, lomustine, leuprolide acetate, vincristine sulfate liposome, procarbazine hydrochloride, mechlorethamine hydrochloride, megestrol acetate, megestrol acetate, trametinib, mercaptopurine, mesna, temozolomide, methotrexate, mitomycin, plerixafor, mechlorethamine hydrochloride, mitomycin c, azacitidine, gemtuzumab ozogamicin, nanoparticle paclitaxel, vinorelbine tartrate, nelarabine, filgrastim, sorafenib tosylate, nilotinib, tamoxifen citrate, romiplostim, ofatumumab, omacetaxine mepesuccinate, pegaspargase, denileukin diftitox, oxaliplatin, paclitaxel, paclitaxel albumin-stabilized nanoparticle formulation, palifermin, palonosetron hydrochloride, pamidronate disodium, panitumumab, pazopanib hydrochloride, pegaspargase, peginterferon alfa-2b, pemetrexed disodium, pertuzumab, cisplatin, plerixafor, pomalidomide, ponatinib hydrochloride, pralatrexate, prednisone, procarbazine hydrochloride, aldesleukin, denosumab, eltrombopag olamine, sipuleucel-t, mercaptopurine, radium 223 dichloride, raloxifene hydrochloride, rasburicas, recombinant interferon alfa-2b, regorafenib, lenalidomide, methotrexate, rituximab, romidepsin, romiplostim, daunorubicin hydrochloride, ruxolitinib phosphat, sipuleucel-t, sorafenib tosylate, dasatinib, regorafenib, peginterferon alfa-2b, thalidomide, omacetaxine mepesuccinate, dabrafenib, tamoxifen citrate, cytarabine, erlotinib hydrochloride, bexarotene, nilotinib, docetaxel, temozolomide, temsirolimus, thalidomide, etoposide, topotecan hydrochloride, toremifene, temsirolimus, tositumomab and i 131 iodine tositumomab, dexrazoxane hydrochloride, trametinib, trastuzumab, bendamustine hydrochloride, lapatinib ditosylate, vandetanib, panitumumab, veip, vinblastine sulfate, bortezomib, vemurafenib, etoposide, leuprolide acetate, azacitidine, vincristine sulfate, vinorelbine tartrate, vismodegib, glucarpidase, vorinostat, pazopanib hydrochloride, leucovorin calcium, crizotinib, capecitabine, denosumab, radium 223 dichloride, enzalutamide, ipilimumab, ziv-aflibercept, vemurafenib, ibritumomab tiuxetan, dexrazoxane hydrochloride, ziv-aflibercept, zoledronic acid, vorinostat, zoledronic acid, and abiraterone acetate.

20. The method of claim 17, wherein the spatial distribution of the cancer cells characterizes the efficacy of the cancer treatment regimen, thereby providing for further treatment decisions or modifications of treatment.
